(19) United States
(12) Patent Application Publication
(10) Pub. No.: US 2020/0232044 A1
(43) Pub. Date: Jul. 23, 2020

(54) TUMOR AND METASTASIZING MARKER

(71) Applicant: Universität zu Köln, Köln (DE)

(72) Inventors: Melanie von Brandenstein, Lindlar (DE); Jochen Fries, Pulheim (DE); Tobias Kohl, Köln (DE); Monika Stefanie Schlosser, Euskirchen (DE); Andreas Stog, Köln (DE)

(73) Assignee: Universität zu Köln, Köln (DE)

(21) Appl. No.: 16/652,827

(22) PCT Filed: Oct. 2, 2018
(86) PCT No.: PCT/EP2018/076761

§ 371 Date: Apr. 1, 2020

(30) Foreign Application Priority Data
Oct. 2, 2017 (EP) 17194448.1

(51) Int. Cl. C12Q 1/6886; C12Q 1/686

(52) U.S. Cl. CPC C12Q 1/6886; C12Q 1/686; C12Q 2600/112; C12Q 2561/113; G01N 2800/60; G01N 2800/7028; C12Q 2600/158

(57) ABSTRACT

The present invention relates to methods for detecting a malignant tumor in an individual by means of determining the Vimentin variant 3 (Vim3) level in the individual's blood serum S and/or the accumulation of Vim3 polypeptide in cell nuclei. Furthermore, the present invention refers to an antineoplastic agent for use in a method for treating an individual bearing a malignant tumor, wherein said antineoplastic agent is a Vim3 inhibitor and/or wherein the malignant tumor is endothelin B receptor negative.

The present invention relates to methods for detecting a malignant tumor in an individual by means of determining the Vimentin variant 3 (Vim3) level in the individual's blood serum S and/or the accumulation of Vim3 polypeptide in cell nuclei. Furthermore, the present invention refers to an antineoplastic agent for use in a method for treating an individual bearing a malignant tumor, wherein said antineoplastic agent is a Vim3 inhibitor and/or wherein the malignant tumor is endothelin B receptor negative.

In clinical contexts, neoplasia are often divided in two major groups: benign neoplasia and malignant tumors. Malignant means that the tumor—having an increased rate of dividing cells (high mitotic rate)—infiltrates healthy surrounding tissue. Secondarily, it infiltrates the walls of blood and/or lymph vessels and into nerve sheaths. Also with vascular infiltration, a malignant tumor has a high potential to shed individual cells or small groups of cells, which are able to disperse in the blood stream or lymph fluid, surviving the cellular host defenses and reaching other organs, where they penetrate the vessels wall to set up satellite tumors, the so-called metastases. On the histologic/cellular level, malignancy is defined by several criteria: changes in nuclear size, form, and genetic content (enlarged, polymorph, and hyperchromatic); the nucleus is bigger than normal in relation to its cellular cytoplasm (an increase in the nuclear to cytoplasmic ratio), and there are cell divisions with atypical arrangements of chromosomes (atypical mitoses). In contrast, the benign neoplasia grows by pushing neighboring tissues leading to pressure induced, cellular atrophy and does not metastasize. In a benign neoplasia, the cellular criteria of malignancy are not observed. One of these neoplasia is the benign oncocytoma such us, e.g., an oncocytome of the kidney.

In clinic, it is of notable benefit to distinguish benign neoplasia from malignant tumors. Further, it is of significant benefit to detect the presence of a malignant tumor as early as possible to improve curability. Malignant tumors are subdivided into six major groups: carcinoma (epithelial), sarcoma (mesenchymal), lymphoma/leukemia (blood cells), neuroendocrine tumors (derived from hormone producing cells), melanomas (malignant skin cells), and tumors derived from the central/peripheral nervous system (astrocytoma, glioblastoma, neurinoma, etc.).

One of the groups bearing high lethality and poor detectability is that of carcinomas. Among carcinomas, several groups of tumors may be distinguished from another based on their origin tissue: those derived from glands (adenocarcinomas), from squamous epithelium such as skin, esophagus, cervix (squamous cell carcinomas), and from urothelium, the lining of urinary tract (urothelial carcinoma or transitional cell carcinoma). Renal cell carcinomas (RCC) are a special group not belonging to any of these mentioned subgroups and thus having extraordinary characteristics.

So far, there are only very rare molecular markers for neoplasia known in the art. In particular, there is still an unmet need for markers that indicate the presence or absence of malignant tumors and that give hindsight for assessing the malignancy, i.e., the propensity to spread metastases, of a malignant tumor.

Vim3 has been described as marker for benign oncocytoma of the kidney, which represents the benign counterpart to malignant renal tumors such as renal cell carcinoma (RCC) (cf., WO 2014/154686; EP17180479.2). Vim3 can be regulated by microRNA-induced methods (von Brandenstein et al., 2018, Oncotarget 9:32855-32867). In the kidney, the malignant renal tumors do not stain to such degree with Vim3. In this context, the kidney is different from the majority of other tissues in that Vim3 levels are herein low in comparison to other tissues, typically even essentially absent in the kidney. An oncocytome leads to increased levels of Vim3 in the renal cells and in the urine. The characteristic cellular staining pattern of an oncocytoma is cytoplasmic. Remarkably, Vim3 is however essentially absent (at least hardly detectable) in blood serum in an individual bearing a benign oncocytoma.

It has now surprisingly been found that individuals bearing a malignant tumor, in particular when the malignant tumor has a high malignancy, the blood serum level of Vim3 is increased. This was particularly found for those individuals bearing a malignant carcinoma, in particular a non-renal carcinoma. Furthermore, the malignant tumor cells (also: cancerous cells, cancer cells) were surprisingly be found to show one or more accumulations of Vim3 in their cell nuclei.

It has now been found that determining the Vim3 level in an individual's blood serum indicates the presence of a malignant tumor, in particular the presence of a tumor having a high propensity to metastasize in the individual.

The present invention refers to a method for detecting a malignant tumor in an individual, said method conducted in vitro comprising determining the Vimentin variant 3 (Vim3) level in the individual's blood serum S. Preferably, herein an increased Vim3 level in the blood serum S indicates the presence of a malignant tumor in the individual.

Therefore, a first aspect of the present invention relates to a method for detecting a malignant tumor in an individual, said method conducted in vitro comprising the following steps:

(i) providing blood serum S obtained from the individual; and
(ii) determining the Vimentin variant 3 (Vim3) level in the blood serum S,
wherein an increased Vim3 level in the blood serum S indicates the presence of a malignant tumor in the individual.

The method of the present invention may be for determining whether the individual bears a malignant tumor, in particular when said individual is suspected for bearing a malignant tumor bearing an increased propensity to metastasize. Such method may be used for routine screening of the individual having or having no previous indication for bearing tumor.

The person skilled in the art will notice that the method of the present invention is preferably a method not directly associated with the diagnosis of the human or animal body. The result obtained by the method may be used for medical or non-medical purposes. The blood serum S typically is an in vitro specimen, i.e., a specimen remote from the human and animal body.

The malignant tumor may be any type of tumor known in the art. In a preferred embodiment, the malignant tumor is malignant carcinoma.

In a more preferred embodiment, the malignant tumor is a malignant carcinoma selected from the group consisting of prostate carcinoma, urothelial carcinoma, bladder carcinoma, transitional cell carcinoma, mucoepidermiod carcinoma, mammacarcinoma, small-cell carcinoma, myoepithelial carcinoma, adenocarcinoma, gastric signet ring cell carcinoma, and esophageal carcinoma.

In a more preferred embodiment, the malignant tumor is a carcinoma except a renal carcinoma, in particular renal cell carcinoma (RCC). In other words, the malignant tumor is preferably a non-renal carcinoma. More preferably, malignant carcinoma is selected from the group consisting of breast adenocarcinoma, small-cell lung adenocarcinoma, prostate adenocarcinoma, mucoepidermoid carcinoma of the salivary glands, and urothelial carcinoma of the bladder. Typically, such designation of tumor refers to the origin tissue of the tumor cells. Therefore, it will be understood that a respective metastasis of such tumor may also nest and thus be located in another tissue surrounding. Tumors of renal and/or gonadal origin, in particular of renal and/or testicular origin, sometimes behave different to other cancer types. Thus, in one embodiment, the neoplasm of interest is preferably not of renal and/or gonadal origin, in particular not renal and/or testicular origin. In one embodiment, the neoplasm of interest is preferably not of renal and/or testicular origin.

Thus, in a preferred embodiment, the neoplasm of interest is a non-gonadal neoplasm. In another preferred embodiment, the neoplasm of interest is a non-renal neoplasm. In another preferred embodiment, the neoplasm of interest is a non-renal, non-gonadal neoplasm. In a highly preferred embodiment, the neoplasm of interest is a non-testicular neoplasm. In a highly preferred embodiment, the neoplasm of interest is a non-renal, non-testicular neoplasm.

In one embodiment, a malignant tumor is preferably not a renal malignant tumor (i.e., preferably is a non-renal malignant tumor). In one embodiment, a carcinoma is preferably not a renal carcinoma (i.e., preferably is a non-renal carcinoma), in particular not renal cell carcinoma (RCC). In one embodiment, a malignant tumor is preferably not a tumor of gonadal origin. In one embodiment, a malignant tumor is preferably not a renal malignant tumor and not a tumor of gonadal origin. In a preferred embodiment, a malignant tumor is preferably not testicular cancer. In a preferred embodiment, a malignant tumor is preferably not Epstein-Barr-Virus (EBV) positive testicular cancer. In a preferred embodiment, a malignant tumor is preferably not an EBV positive tumor. In a preferred embodiment, a malignant tumor does not comprise increased contents of microRNA that downregulates ET-1 and/or Vim3.

In a preferred embodiment, the tumor of interest is a non-gonadal (malignant) tumor. In another preferred embodiment, the (malignant) tumor of interest is a non-renal (malignant) tumor. In another preferred embodiment, the tumor of interest is a non-renal (malignant), non-gonadal (malignant) tumor. In a highly preferred embodiment, the tumor of interest is a non-testicular (malignant) tumor. In a highly preferred embodiment, the tumor of interest is a non-renal, non-testicular (malignant) tumor.

In one preferred embodiment, the malignant cancer of which the tumor is of has a regression rate of <70% or <60%.

Vim3 (also designated as Vimentin3, Vimentin variant 3, Vimentin splice form 3) is a splice isoform of Vimentin. Vim3 in the context of the present invention may be any Vim3 compound. Vimentin itself is an intermediate sized filament that functions in signal transduction cellular function, structural integrity of cells and tissues and adhesion and migration. In 2007, a variant of Vimentin (Vim3) was described by a working group of the Craig Venter Institute (NHLBI Resequencing and Genotyping Service (RSG), N01-NV-48196, J. Craig Venter Institute, Rockville, Md. 20850). In 2011, the presence of this Vim3 in gliomas was described (Thakkar et al., 2011, Cancer Invest 29:113-122). Vim3 is a spliced variant of Vimentin with a unique C-terminal ending. Preferably, Vim3 is the naturally occurring Vim3 of the species of the individual of interest, i.e., the Vim3 occurring in the respective blood serum S. Exemplarily, Vim3 may be human Vim3 or Vim3 of a non-human animal, in particular a domestic mammal such as, e.g., a bovine, a pig, a horse, a donkey, a sheep, a goat, a dog, a cat, etc.). Vim3 has been found in numerous species so far. In a particularly preferred embodiment, Vim3 is human Vim3. Vimentin is an intermediate-sized filament that functions in cellular signal transduction, structural integrity of cells and tissues, as well as adhesion and migration (Lowery et al., 2015, Intermediate Filaments Play a Pivotal Role in Regulating Cell Architecture and Function. J Biol Chem. 2015; 290(28):17145-53. (Review)). This full-length molecule is detected in the cytoplasm of cells and in the matrix in between cells as mesenchymal component. The genomic sequence of full-length Vimentin contains 9 exons with a protein size of about 57 kDa, being structured in a head, rod and tail domain (Quax et al., 1983, The structure of the vimentin gene. Cell. 1983; 35:215-223). The tail domain is responsible for cytoplasmic compartmentalization (Rogers et al., 1995, Truncation mutagenesis of the non-alpha-helical carboxyterminal tail domain of vimentin reveals contributions to cellular localization but not to filament assembly. Eur J Cell Biol. 1995, 66:136-150); meaning it does not migrate into the nucleus.

In contrast, in 2007 a spliced variant of Vimentin (called Vim3) with a unique C-terminal ending was detected by a working group at the Craig Venter Institute (NHLBI Resequencing and Genotyping Service (RSG), N01-NV-48196, J. Craig Venter Institute, Rockville, Md. 20850). Vim3 lacks exons 8 and 9 resulting in a protein of only 47 kDa. While Vim3 was observed in the cytoplasm of oncocytoma (in particular those of the kidney), in malignant tumors if it is detected, is predominantly found in the nucleus. In a pathophysiological context, this observation can be explained by experimental data showing that tail-deleted vimentin is able to migrate into the nucleus (Eckelt et al., 1992, Assembly of a tail-less mutant of the intermediate filament protein, vimentin, in vitro and in vivo. Eur J Cell Biol. 1992; 58:319-330). Furthermore, only the rod domain is responsible for the potential of nuclear migration while headless vimentin remains restricted to the cytoplasm due to its tail domain (Rogers et al., 1995, Truncation mutagenesis of the non-alpha-helical carboxyterminal tail domain of vimentin reveals contributions to cellular localization but not to filament assembly. Eur J Cell Biol. 1995, 66:136-150).

The human splice variant is typically 35 amino acids smaller than the full length protein. Its unique structure leads to a 10 kDa smaller protein. The amino acid sequence of human Vim3 has been published and is available at UniProt KB (http://www.uniprot.org/uniprot/B0YJC4) or at the National Center for Biotechnology Information under GenBank Accession number ACA06103.1 (http://www.ncbi.nlm.nih.gov/protein/167887751). Preferably, in the context of the present invention, Vim3 has a homology of at least 80%, more preferably at least 90%, even more preferably at least 95%, even more preferably at least 98%, even more preferably at least 99% of SEQ ID NO: 1, in particular is identical with SEQ ID NO: 1:

| | |
|---|---|
| 1 | mstrsvssss yrrmfggpgt asrpsssrsy vttstrtysl gsalrpstsr slyasspggv |
| 61 | yatrssavrl rssvpgvrll qdsvdfslad aintefkntr tnekvelqel ndrfanyidk |
| 121 | vrfleqqnki llaeleqlkg qgksrlgdly eeemrelrrq vdqltndkar veverdnlae |
| 181 | dimrlreklq eemlqreeae ntlqsfrqdv dnaslarldl erkveslqee iaflkklhee |
| 241 | eiqelqaqiq eqhvqidvdv skpdltaalr dvrqqyesva aknlqeaeew ykskfadlse |
| 301 | aanrnndalr qakqesteyr rqvqsltcev dalkgtnesl erqmremeen faveaanyqd |
| 361 | tigrlqdeiq nmkeemarhl reyqdllnvk maldieiaty rkllegeesr islplpnfss |
| 421 | lnlrgkhfis l |

The following primer may be used to obtain the Vim3:

Forward:
(SEQ ID NO: 4)
5′-GAGAACTTTGCCGTTGAAGC-3′
Reverse:
(SEQ ID NO: 5)
5′-GAAATAAAATGCTTACCCCTCAG-3′

In an alternative preferred embodiment, Vim3 is mammalian non-human Vim3 such as, e.g., bovine Vim3, pig Vim3, horse Vim3, donkey Vim3, sheep Vim3, goat Vim3, dog Vim3, or cat Vim3. The Vimentin sequence of other species is also known, including e.g. Mus musculus (NCBI Accession: CAA39807.1, NP_035831.2), Rattus norvegicus (NCBI Accession: NP_112402.1), Bos taurus (NCBI Accession: NP_776394.2), Gallus gallus (NCBI Accession: NP_001041541.1), Mesocricetus auratus (Accession: AAA37104.1), Oncorhynchus mykiss (Accession: CAA90601.1), Equus caballus (NP_001230074.1), Salmo salar (Accession: NP_001133947.1), Pan troglodytes (Accession: NP 001009148.1) and Cavia porcellus (Accession: NP_001166511.1). The splice variant corresponding to human Vim3 could be easily identified by sequence analysis and identification of homologues.

As used herein, the term “Vim3 level” may be understood in the broadest sense, including the Vim3 polypeptide level and/or the Vim3 mRNA level (directly correlating to the Vim3 polypeptide level) in a sample (e.g., the blood serum S or a control sample). The Vim3 level may also be understood as Vim3 expression level.

As used herein, the term “level” may be understood in the broadest sense as an content of a respective polypeptide or messenger RNA (mRNA) in a sample (e.g., the blood serum S or a control sample). Typically, but not necessarily, such level is related to a reference value such as the sample volume, the total polypeptide or nucleotide content comprised in same sample (e.g., the blood serum S or a control sample) or the content of an intrinsic marker (e.g., transferrin, beta-actin, and/or serum albumin). If related to the sample volume, the level indicates the concentration, i.e., the respective polypeptide or mRNA per volume (e.g, mass of Vim3 polypeptide per volume of the blood serum S [ng/ml]). If related to the total polypeptide content, a relative ratio may be provided (e.g, mass of Vim3 polypeptide per mass of total polypeptide content of the blood serum S [pg/ng]). If related to the total nucleotide, a relative ratio may be provided (e.g, mass of Vim3 mRNA per mass of total nucleotide content of the blood serum S [pg/ng]). If related to the content of an intrinsic marker, a relative ratio may be provided (e.g, mass of Vim3 polypeptide per mass of transferrin [pg/pg]).

Nuclear transmigration is characteristic for proteins which modulate transcription at the genomic level, so called transcription factors either by binding directly themselves to specific recognition sites on the promoter sequence due to complementary nucleotide sequences or by forming a specific complex with several other factors (proteins), of which one of its participants interacts with the genomic sequence to induce transcription. Vim can obviously act as a transcription factor.

Using immunoprecipitation and protein immunoblotting, Vim3 could be shown to sit within such a complex together with autophagy-related protein 7 (Atg7) and tumor protein p53 (p53, also: cellular tumor antigen p53, phosphoprotein p53, tumor suppressor p53, antigen NY-CO-13, and transformation-related protein 53 (TRP53)). This complex was identified in the nuclei of malignant prostate adenocarcinoma cells in which Vim3 can be detected either by immunoblotting of nuclear extracts or by fluorescent immune histology with a specific anti-Vim3 antibody. Analyzing the underlying signaling mechanism, nuclear extracts were prepared from resting and proliferating prostate cancer cells and individual protein components captured through immunoprecipitation using specific antibodies attached to agarose beads. The components were subsequently immunoblotted by Western blot. Doing so, Vim3 and Atg7 could be found building a complex with p53 in the nucleus (cf. FIG. 1). Such Vim3/Atg7/p53 complexes particularly occur when the signal transduction of the endothelin B receptor (ETBR) is blocked and the signal of endothelin-1 (ET-1) particularly effectively triggers the endothelin A receptor (ETAR). This is schematically depicted in FIG. 1 herein.

P53 is a protein known to regulate a wide variety of cellular functions including apoptosis, cell cycle, DNA repair and metabolism. The full length protein consists of 393 amino acids. Over 40 isoforms are known (Jimxiong et al., 2012) created by alternative splicing and the use of different promoters, which in a tissue specific manner are detectable besides the full length protein. Thus, in prostate cancer p63 can be found together with p53 in the cytoplasm (Dhillon et al., 2009, Aberrant cytoplasmic expression of p63 and prostate cancer mortality, Cancer Epidemiology Biomarkers and Prevention, vol. 18, no. 2, pp. 595-600, 2009).

Atg7 is considered the essential autophagy gene, being a member of a class of 18 autophagy-related (ATG) genes, which are essential for autophagosome formation (Mizushima et al., 2011, The role of Atg proteins in autophagosome formation. Annu Rev Cell Dev Biol. 2011; 27:107-32). Atg7 has been described to form a complex with p53 (Lee et al., 2012, Atg7 modulates p53 activity to regulate cell cycle and survival during metabolic stress. Atg7 modulates p53 activity to regulate cell cycle and survival during metabolic stress. Science. 2012 Apr. 13; 336:225-228; Kageyama and Komatsu, 2012) in the cytoplasm. Via its nuclear location sequence, p53 is bound by importin alpha, which as chaperone protein transports the entire complex into the nucleus (Berglund et al., 2008, A genecentric Human Protein Atlas for expression profiles based on antibodies. Mol Cell Proteomics. 2008; 7:2019-27). Here, these factors can act in concert as transcription factors, binding to promoter regions such as the Cdknla, a cyclin-dependent kinase inhibitor (Lee et al., 2012, Atg7 modulates p53 activity to regulate cell cycle and survival during metabolic stress. Atg7 modulates p53 activity to regulate cell cycle and survival during metabolic stress. Science. 2012 Apr. 13; 336:225-228).

Autophagy is a pathway for lysosomal degradation by which it can regulate tumorigenesis (Kenific and Debnath, 2015, Cellular and metabolic functions for autophagy in cancer cells. Trends Cell Biol. 2015; 25:37-45) by maintenance of metabolic pathways important for aggressive tumor growth. The suggested mechanism is according to literature that defects in mitochondria metabolism compromise tumor growth. Thus in several experimental models of Atg7 deficiency, progression of adenocarcinomas to benign oncocytomas has been described, of which all being defective in their respiratory chain. Towards that end, deletion of Atg7 has been produced in genetically engineered mice of different carcinomas, showing delay in tumor progression [KRAS-driven human non-small cell lung cancer (Guo et al., 2013, Autophagy suppresses progression of K-ras-induced lung tumors to oncocytomas and maintains lipid homeostasis. Genes Dev. 2013; 27:1447-1461); mouse model of pancreatic ductal adenocarcinoma (Rosenfeldt et al., 2013, p53 status determines the role of autophagy in pancreatic tumour development. Nature. 2013; 504(7479):296-300); Atg7 (−/−) mice are protected from pre-cancerous colonic lesions (Levy et al., 2015, Intestinal inhibition of Atg7 prevents tumour initiation through a microbiome-influenced immune response and suppresses tumour growth. Nat Cell Biol. 2015; 17:1062-1073); Atg7 (−/−) together with a PTEN-deficiency (Santanam et al., 2016, Atg7 cooperates with Pten loss to drive prostate cancer tumor growth. Genes Dev. 2016; 30:399-407). These data support the observation that patients with tumors overexpressing Atg7/p53 have a worse prognosis (Kenific and Debnath, 2015, Cellular and metabolic functions for autophagy in cancer cells. Trends Cell Biol. 2015; 25:37-45). This has been observed in several different human cancers, among them breast cancer (Desai et al., 2013, Heat shock factor 1 (HSF1) controls chemoresistance and autophagy through transcriptional regulation of autophagy-related protein 7 (ATG7). J Biol Chem. 2013 March; 288:9165-9176), bladder carcinoma (Lin et al., 2016, Inhibition of High Basal Level of Autophagy Induces Apoptosis in Human Bladder Cancer Cells. J Urol. 2016; 195:1126-1135), and esophageal carcinoma (Zhu et al., 2013, ATG7 deficiency promote apoptotic death induced by Cisplatin in human esophageal squamous cell carcinoma cells. Bull Cancer. 2013; 100:15-21).

Consequently to showing by immunoprecipitation from nuclear extracts of malignant tumor cells such as prostate adenocarcinoma that Vim3 is part of transcriptional complex (in particular a Vim3/Atg7/p53 complex) migrating in the nucleus, it appears highly probable that the detection of nuclear Vim3 staining in malignant cancers can serve as biomarker for tumor progression.

Its transcriptional function is highly probable: Burkhari et al. (2015) describe—without differentiating between Vim3 and its full length version—that vimentin binds the ALOX15 promoter and regulates its promoter activity, again strongly suggesting an active role as transcription factor. Besides being a marker for immunhistologic evaluation of tumor slides, Vim3 could serve as a biomarker for serological screening. By affinity proteomic studies, Burkhari et al. (2015) identified vimentin as a potential biomarker in colon cancers from serological screening and computational modelling. It has been surprisingly shown that using a Vim3 ELISA that Vim3 serum levels are increased in urothelial carcinoma, bladder carcinoma, and prostate carcinoma vs. non-tumor control patients (cf., FIGS. 2 and 3).

The blood serum S may be obtained by any means. Preferably, it is obtained by obtaining a blood sample of the individual of interest of which the blood cells and the clotting factors have been removed. The method of the present invention may be conducted in fresh blood serum (e.g., by contacting an ELISA or via a dipstick with the blood serum S in a vessel/hollow ware) or may be conducted using a stored blood serum S or blood serum S obtained from stored blood. Storage may be storage of up to 15 min (minutes), up to 30 min, up to one hour, up to twelve hours, up to a day, up to a week, up to a month, up to a year or even longer. Long-term storage for more than one day is preferably conducted under any conditions maintaining detectability of the level of the Vim3 polypeptide such as, e.g., by means of freezing, shock-freezing (e.g., in liquid nitrogen), freeze-draying, and/or the addition of one or more preservative agents, in particular biocide/antimicrobial agents, to the blood serum S.

An individual of interest from which the blood serum S is obtained from may be a human or non-human animal. A non-human animal preferably is a non-human mammal, in particular a domestic mammals such as, e.g., a bovine, a pig, a horse, a donkey, a sheep, a camel, a goat, a dog, a cat, etc. Preferably, the individual of interest from which the blood serum is obtained from is a human. The individual, in particular when it is a human, may also be designated as patient.

Endothelin-1 (ET-1) is a vital agent in the growth and progression of several tumors including prostate, ovarian, colorectal, bladder, breast and lung carcinomas. ET-1 exerts its effects through the activation of two distinct receptors, ETAR and ETBR (Bhalla et al., 2009, Endothelin receptor antagonism and cancer. Eur J Clin Invest. 2009; 39 Suppl 2:74-7). In prostate carcinoma cells, downregulation of ETBR channels signaling via the ETAR. It was surprisingly found that this leads to increased proliferation of tumor cells indicated by closing the cell gap in a wounding assay. When using a fluorescent labeled antibody to Vim3, one can demonstrate a migration from the cytoplasm in resting cells to the nucleus in proliferating cells closing the wound (cf., FIG. 13). Thus Vim3 is a marker for tumor proliferation which is a prerequisite of tumor progression.

It is assumed that in benign oncocytoma cell signaling is different and other complexes comprising Vim3 are formed that do not base on complexes of Vim3 with Atg7 and p53.

As used herein, an oncocytoma may be understood in the broadest sense. Preferably, it is an oncocytoma of renal origin, in particular an oncocytoma of renal origin present in the kidney.

As indicated above, Vim3 may also be used as metastasizing marker. Accordingly, in a preferred embodiment, an increased Vim3 level in the blood serum S indicates the presence of one or more metastases in the individual, the propensity of the tumor to metastasize, or a combination of both.

In other words, an aspect of the present invention relates to a method for detecting a malignant tumor in an individual, said method conducted in vitro comprising the following steps:

(i) providing blood serum S obtained from the individual; and
(ii) determining the Vim3 level in the blood serum S,
wherein an increased Vim3 level in the blood serum S indicates the presence of one or more metastases in the individual, the propensity of the tumor to metastasize, or a combination of both.

In a highly preferred embodiment, the present invention relates to a method for detecting a malignant carcinoma in an individual, said method conducted in vitro comprising the following steps:

(i) providing blood serum S obtained from the individual; and
(ii) determining the Vim3 level in the blood serum S,
wherein an increased Vim3 level in the blood serum S indicates the presence of one or more metastases in the individual, the propensity of the carcinoma to metastasize, or a combination of both.

In a particularly preferred embodiment, the present invention relates to a method for detecting a malignant carcinoma in an individual, said method conducted in vitro comprising the following steps:

(i) providing blood serum S obtained from the individual; and
(ii) determining the level of Vim3 polypeptide in the blood serum S,
wherein an increased level of Vim3 polypeptide in the blood serum S indicates the presence of one or more metastases in the individual, the propensity of the carcinoma to metastasize, or a combination of both.

An aspect of the present invention relates to a method for detecting one or more metastases in an individual and/or for determining the propensity of the tumor to metastasize in an individual, said method conducted in vitro comprising the following steps:

(i) providing blood serum S obtained from the individual; and
(ii) determining the Vim3 level in the blood serum S,
wherein an increased Vim3 level in the blood serum S indicates the presence of one or more metastases in the individual, the propensity of the tumor to metastasize, or a combination of both.

An aspect of the present invention relates to a method for detecting one or more metastases in an individual, said method conducted in vitro comprising the following steps:

(i) providing blood serum S obtained from the individual; and
(ii) determining the Vim3 level in the blood serum S,
wherein an increased Vim3 level in the blood serum S indicates the presence of one or more metastases in the individual.

An aspect of the present invention relates to a method for determining the propensity of the tumor to metastasize in an individual, said method conducted in vitro comprising the following steps:

(i) providing blood serum S obtained from the individual; and
(ii) determining the Vim3 level in the blood serum S,
wherein an increased Vim3 level in the blood serum S indicates the propensity of the tumor to metastasize.

In view of the above, the outcome of the method of the present invention may have a significant influence on the further treatment strategy. It may however also be used for scientific purposes only.

In principle, a method as claimed does not need any control samples. On the one hand the experimenter conducting the method will soon recognize certain thresholds indicating the borderline between blood serum indicating the presence of a malignant tumor and a blood serum of the same species indicating the absence of a malignant tumor. This value may be determined for each device used.

As used herein, the terms “borderline” and “threshold” may be understood interchangeably in the broadest sense as a value that separates two groups from another.

Secondly, when measuring a large number of samples concomitantly, today, the intrinsic “control” is the average of values. Those levels significantly deviating from the average indicate values of relevance, i.e., patient that bear special characteristics such as one or more malignant tumors and/or metastases.

In order to improve comparability of different blood serums S with another, in other words to normalize the determined and to improve reproducibility of the measurements, the determined Vim3 level determined in step (ii) may be compared to a reference value. Such reference value may be an internal control (i.e., a further control sample C measured under comparable conditions, preferably in the same test series) or may be a predetermined reference value R1 typically but not necessarily obtained from one or more previous measurements conducted under comparable conditions.

Thus, in a preferred embodiment, the method further comprises step (iii) of comparing the Vim3 level determined in step (ii) with a predetermined reference value R1 indicating the borderline between blood serum indicating the presence of a malignant tumor and a blood serum of the same species indicating the absence of a malignant tumor,

wherein an Vim3 level determined in the blood serum S that is higher than R1 indicates the presence of a malignant tumor in the individual, and
wherein the Vim3 level is related to the total polypeptide content comprised in the respective sample.

In a preferred embodiment, a Vim3 level determined in step (ii) above a borderline indicates a higher risk or the presence of a malignant tumor.

Such borderline may be in the range that separates the bulk of the concentration ranges of Vim3 levels in sera of individuals of higher risk or the presence of a malignant tumor from the bulk of the concentration ranges of Vim3 levels in sera of individuals of lower risk.

In this context the bulk may be understood in the broadest sense as a group containing the majority of concentration ranges of individual's sera (typically >50% by number). In a preferred embodiment, the distribution of the Vim3 levels are calculated for both groups (i.e., higher risk or the presence of a malignant tumor versus lower risk) and the borderline is the Vim3 level where the distribution ranges cross. In a preferred embodiment, the distribution is fitted by a Gaussian distribution (standardized normal distribution). Then, the borderline is the Vim3 level where the Gaussian distributions (Gaussian curves) cross.

A borderline as described above may depend on the nature of the neoplasm of interest and on the experimental conditions. In one example, such borderline may be in the range of between 100 and 600 ng/ml, between 200 and 500 ng/ml, between 220 and 400 ng/ml, between 250 and 350 ng/ml, between 300 and 350 ng/ml, or between 250 and 300 ng/ml Vim3 in blood serum.

In a still further embodiment, the distribution is characterized by a still further, second borderline, separating the group of lower risk in further groups such as having a very low risk versus having some risk.

In one example, the case of prostate cancer (in particular prostate carcinoma), the borderline may be in the range of 350 ng/ml in blood serum. Thus, a Vim3 level above 350 ng/ml may be indicative for a rather high risk of malignant prostate cancer (in particular prostate carcinoma). A Vim3 level of 320 to 350 ng/ml in blood serum may be indicative for a still notable risk and a Vim3 level below 320 nm may indicate a rather low risk. As used in the context of prostate cancer (in particular prostate carcinoma), low risk means those individuals having a Gleason Scale of (Gleason) 6. Individuals of intermediate risk are those with Gleason 7a and 7b. Individuals of high risk are those with Gleason 8, 9 or 10. Metastasizing prostate cancer is often under therapeutic treatment. Therefore, a Vim3 level above 350 ng/ml in blood serum may indicate Gleason 8, 9 or 10.

In the case of urothel carcinoma, in one example, the borderline may be in the range of 300 ng/ml in blood serum. Thus, a Vim3 level above 300 ng/ml in blood serum may be indicative for a rather high risk of malignant urothel carcinoma. A Vim3 level of 250 to 300 ng/ml in blood serum may be indicative for a still notable risk and a Vim3 level below 250 nm may indicate a rather low risk.

Borderlines in a comparable range are found in pancreas carcinoma and other cancer types, in particular carcinoma. It will however, also be noted that the person skilled in the art can determine borderline values for any neoplasms without any burden by means of comparing different risk groups with another.

In an additional or alternative preferred embodiment, the method further comprises step (iii) of comparing the Vim3 level determined in step (ii) with a Vim3 level determined in a control sample C obtained from one or more control individuals of the same species free of a malignant tumor,

wherein an Vim3 level determined in the blood serum S that is at least 10%, preferably 2 at least 20%, in particular at least 30%, higher than the Vim3 level of C indicates the presence of a malignant tumor in the individual, and
wherein the Vim3 level is related to the total polypeptide content comprised in the respective sample.

In the context of the present invention, the terms “polypeptide” and “protein” may be understood interchangeably in the broadest sense as a compound mainly composed of natural amino acid moieties consecutively conjugated with another via amide bonds. It will be understood that a protein in the sense of the present invention may or may not be subjected to one or more posttranslational modification(s) and/or be conjugated with one or more non-amino acid moiety/moieties. The termini of the protein may, optionally, be capped by any means known in the art, such as, e.g., amidation, acetylation, methylation, acylation. Posttranslational modifications are well-known in the art and may be but may not be limited to lipidation, phosphorylation, sulfatation, glycosylation, truncation, oxidation, reduction, decarboxylation, acetylation, amidation, deamidation, disulfide bond formation, amino acid addition, cofactor addition (e.g., biotinylation, heme addition, eicosanoid addition, steroid addition) and complexation of metal ions, non-metal ions, peptides or small molecules and addition of iron-sulphide clusters. Moreover, optionally, co-factors, in particular cyclic guanidinium monophosphate (cGMP), but optionally also such as, e.g., ATP, ADP, NAD⁺, NADH+H⁺, NADP⁺, NADPH+H⁺, metal ions, anions, lipids, etc. may be bound to the protein, irrespective on the biological influence of these co-factors. It will be understood that such polypeptide may also bear one or more non-natural amino acid moiety/moieties and/or one or more posttranscriptional modification(s) and/or may be conjugated to one or more further structures such as label moieties (e.g., by means of a dye (e.g., a fluorescence dye) or a metal label (e.g., gold beads)). In the context of Vim3, the respective polypeptide is preferably the one generated by the individual of interest.

Preferably, the level of the Vim3polypeptide is conducted by determining the level directly in the blood serum S. Alternatively, an aliquot of the blood serum S may be diluted in a liquid that maintains the detectability of the levels of the Vim3 polypeptide (e.g., by means of an aqueous buffer and/or an organic solvent (e.g., dimethyl sulfoxide)).

The step of determining Vim3 levels in the blood serum S may be conducted by any means. Preferably, the step of determining the Vim3 level is determining the level of Vim3 polypeptide and/or the level of Vim3 messenger RNA (mRNA), in particular the Vim3 polypeptide. Determining the level of a polypeptide typically bears the advantage that polypeptides are rather stable in body fluids such as blood. Therefore, the level of Vim3 polypeptide can be determined in the blood serum S without any burden.

Accordingly, in a preferred embodiment, the step (ii) of determining the Vim3 level is determining the level of Vim3 polypeptide.

In order to improve comparability of different blood serums S with another, in other words to normalize the determined results, the level of Vim3 polypeptide determined in a blood serum S is preferably related to a reference value such as the sample volume, the total polypeptide content comprised in same sample (e.g., the blood serum S or a control sample) or the content of an intrinsic marker (e.g., albumin, transferrin and/or beta-actin) of known concentration naturally contained in the same sample (e.g., the blood serum S or a control sample). If related to the sample volume, the level indicates the concentration, i.e., the respective polypeptide per volume (e.g, mass of Vim3 polypeptide per volume of the blood serum S [ng/ml]). If related to the total polypeptide content, a relative ratio may be provided (e.g, mass of Vim3 polypeptide per mass of total polypeptide content of the blood serum S [pg/ng]). If related to the content of an intrinsic marker, a relative ratio may be provided (e.g, mass of Vim3 polypeptide per mass of transferrin [pg/pg] or the mass of Vim3 polypeptide per mass of beta-actin [pg/pg]) or the mass of Vim3 polypeptide per mass of albumin [pg/ng]).

Highly preferably, the step of determining the Vim3 level is determining the level of Vim3 polypeptide in relation to the total polypeptide content comprised in the respective sample.

In a highly preferred embodiment, the step (ii) of determining the Vim3 level is determining the level of Vim3 polypeptide by means of conducting at least one step selected from the group consisting of enzyme-linked immunosorbent assay (ELISA), immuno-electrophoresis, immuno-blotting, Western blot, SDS-PAGE, capillary electrophoresis (CE), spectrophotometry or enzyme assay for example, dipsticks (lateral flow), and combinations of two or more thereof.

In a preferred embodiment, the step (ii) of determining the Vim3 level is determining the level of Vim3 polypeptide and wherein said step (ii) comprises staining of the Vim3 polypeptide, preferably by means of:
(iia) direct immunodetection comprising providing at least one labeled antibody or antibody fragment AB1-L specific for the Vim3 polypeptide, and enabling the binding of said AB1-L to the Vim3 polypeptide; or
(iib) indirect immunodetection comprising providing at least one unlabeled antibody or antibody fragment AB1-ul specific for the Vim3 polypeptide and at least one labeled antibody or antibody fragment AB2-L specifically binding to AB1-ul, enabling the binding of AB1-ul to the Vim3 polypeptide, and enabling the binding of AB2-L to AB1-ul.

Particularly preferably, step (ii) comprises at least one of determining the Vim3 polypeptide level by means of conducting at least one step selected from the group consisting of enzyme-linked immunosorbent assay (ELISA), immuno-electrophoresis, immuno-blotting, Western blot, SDS-PAGE, capillary electrophoresis (CE), spectrophotometry or enzyme assay for example, dipsticks (lateral flow), and combinations of two or more thereof.

In a particularly preferred embodiment, step (ii) comprises at least one of determining the Vim3 polypeptide level by means of an enzyme-linked immunosorbent assay (ELISA). Then, plates used for this purpose may be washed (e.g., by phosphate buffered saline (PBS)) and incubated with serum pf the individual of interest (patient) and subsequently again washed (e.g., by phosphate buffered saline (PBS)) in order to remove unbound protein. The plate may then be incubated with a vimentin (Vim3-specific antibody (e.g., Vimentin 3B4 antibody, and antibody as exemplified below etc.) and again washed (e.g., by phosphate buffered saline (PBS)) in order to remove unbound antibody. Then, a secondary antibody (e.g., such binding to the Fc part of the vimentin/Vim3-specific antibody, such as, e.g., an anti-rabbit antibody) and again washed (e.g., by phosphate buffered saline (PBS)) in order to remove unbound antibody. This secondary antibody may be conjugated to an enzyme (e.g., peroxide). Exemplarily, a 3,3′,5,5′-Tetramethylbenzidin (TMB solution) may be used for staining purposes. A respective enzymatic label will be chosen. The reacting may finally be stopped (e.g., TMB solution by means of addition of sulfuric acid). The reaction products may be detected at a respective wavelength (e.g., in case of a TMB, of 495 nm). For the quantification and control, a high control (e.g., a serum of an individual bearing at least one malignant tumor) and a low control (e.g., a serum of a healthy individual) be optionally loaded on the same plate.

Preferred characteristics of a Vim3-specific antibody or fragment or variant thereof are described in WO 2014/154686. Preferably, in the context of Vim3, the antibody or antibody fragment binds to Vim3 with an at least 10-fold, even more preferably at least 100-fold, even more preferably at least 1000-fold higher binding affinity than to full-length Vimentin (V9). In a particularly preferred embodiment, the antibody or antibody fragment binds to the unique C-terminal 8 amino acids of Vim3 (RGKHFISL: SEQ ID No: 2) and/or unique C-terminal 10 amino acids of Vim3 (NLRGKHFISL: SEQ ID NO: 3). This is further exemplified in WO 2014/154686 and in the example section.

In a preferred embodiment, the specific antibody may be obtained from (Vim3 antibody, EZBiolab, Inc. Carmel, USA, Vimentin V9, Santa Cruz, Heidelberg, Germany, AMACR and CD117, Dako, Hamburg, Germany). Then, the secondary antibody binding to the first, specific antibody may be an anti-rabbit antibody (e.g., obtained from Santa Cruz, Heidelberg, Germany, that may be FITC-labeled).

As used in the context of the present invention, the term “antibody” may be understood in the broadest sense as any type of immunoglobulin or antigen-binding fraction or variant thereof known in the art. Exemplarily, the antibody of the present invention may be an immunoglobulin A (IgA), immunoglobulin D (IgD), immunoglobulin E (IgE), immunoglobulin G (IgG), immunoglobulin M (IgM), immunoglobulin Y (IgY) or immunoglobulin W (IgW). Preferably, the antibody is an IgA, IgG or IgD. More preferably, the antibody is an IgG. However, it will be apparent that the type of antibody may be altered by biotechnological means by cloning the gene encoding for the antigen-binding domains of the antibody of the present invention into a common gene construct encoding for any other antibody type.

The binding between the antibody and its molecular target structure (i.e., its antigen, Vim3) typically is a non-covalent binding. Preferably, the binding affinity of the antibody to its antigen has a dissociation constant (Kd) of less than 1 μM, less than 500 nM, less than 200 nM, less than 100 nM, less than 50 nM, less than 40 nM, less than 30 nM or even less than 20 nM.

Preferably, the binding affinity to Vim3 is higher than to full length MAPK p38. In a preferred embodiment, the antibody or fragment or variant thereof binds to the Vim3 polypeptide with a dissociation constant of not more than 20 nM and, preferably, to full length vimentin with a dissociation constant of more than 20 nM.

The term “antibody” as used herein may be understood in the broadest sense and also includes what may be designated as an antibody variant (also: antibody mutant). As used in the context of the present invention, the terms “antibody variant” and “antibody mutant” may be understood interchangeably in the broadest sense as any antibody mimetic or antibody with altered sequence known in the art. The antibody variant may have at least 10%, at least 20%, at least 50%, at least 60%, at least 70%, at least 80%, at least 85%, at least 90% or at least 95% of the binding affinity of a corresponding antibody, i.e., bear a dissociation constant (Kd) of less than 10 μM, less than 1 μM, less than 500 nM, less than 200 nM, less than 100 nM, less than 50 nM, less than 40 nM, less than 30 nM or even less than 20 nM.

As used herein, the term “antibody fragment” may be understood in the broadest sense as any fragment of an antibody that still bears binding affinity to its molecular target (i.e., its antigen, e.g., Vim3). Exemplarily, the antibody fragment may be a fragment antigen binding (Fab fragment), Fc, F(ab′)₂, Fab′, scFv, a truncated antibody comprising one or both complementarity determining region(s) (CDR(s)) or the variable fragment (Fv) of an antibody. Variable domains (Fvs) are the smallest fragments with an intact antigen-binding domain consisting of one V_{L} and one V_{H}. Such fragments, with only the binding domains, can be generated by enzymatic approaches or expression of the relevant gene fragments, e.g. in bacterial and eukaryotic cells. Different approaches can be used, e.g. either the Fv fragment alone or ‘Fab’-fragments comprising one of the upper arms of the “Y” that includes the Fv plus the first constant domains. These fragments are usually stabilized by introducing a polypeptide link between the two chains which results in the production of a single chain Fv (scFv). Alternatively, disulfide-linked Fv (dsFv) fragments may be used. The binding domains of fragments can be combined with any constant domain in order to produce full length antibodies or can be fused with other polypeptides and polypeptides. A recombinant antibody fragment is the single-chain Fv (scFv) fragment. Dissociation of scFvs results in monomeric scFvs, which can be complexed into dimers (diabodies), trimers (triabodies) or larger aggregates such as TandAbs and Flexibodies. The antibody may be a Fab, a Fab′, a F(ab′)2, a Fv, a disulfide-linked Fv, a scFv, a (scFv)₂, a bivalent antibody, a bispecific antibody, a multispecific antibody, a diabody, a triabody, a tetrabody or a minibody.

As mentioned above, the term “antibody” may also include an antibody mimetic which may be understood in the broadest sense as organic compounds that, like antibodies, can specifically bind antigens and that typically have a molecular mass in a range of from approximately 3 kDa to approximately 25 kDa. Antibody mimetics may be, e.g., affibody molecules (affibodies), aptamers, affilins, affitins, anticalins, avimers, DARPins, Fynomers, Kunitz domain peptides, single-domain antibodies (e.g., VHH antibodies or VNAR antibodies, nanobodies), monobodies, diabodies, triabodies, flexibodies and tandabs. The antibody mimetics may be of natural origin, of gene technologic origin and/or of synthetical origin. The antibody mimetics may also include polynucleotide-based binding units. Optionally, the antibody may also be a CovX-body. Optionally, the antibody may also be a cameloid species antibody.

The antibody according to the present invention is preferably a monoclonal antibody, a chimeric antibody or a humanized antibody. Monoclonal antibodies are monospecific antibodies that are identical because they are produced by one type of immune cell that are all clones of a single parent cell. A chimeric antibody is an antibody in which at least one region of an immunoglobulin of one species is fused to another region of an immunoglobulin of another species by genetic engineering in order to reduce its immunogenicity. For example murine V_{L} and V_{H} regions may be fused to the remaining part of a human immunoglobulin. A particularly preferred type of chimeric antibodies are humanized antibodies. Humanized antibodies are produced by merging the DNA that encodes the CDRs of a non-human antibody with human antibody-producing DNA. The resulting DNA construct can then be used to express and produce antibodies that are usually not as immunogenic as the non-human parenteral antibody or as a chimeric antibody, since merely the CDRs are non-human.

The antibody or antibody fragment, independent on its chemical nature, may optionally be dissolved in any medium suitable for storing said antibody such as, e.g., water, an aqueous buffer (e.g., a Hepes, Tris, or phosphate buffer (e.g. phosphate buffered saline (PBS)), an organic solvent (e.g., dimethyl sulfoxide (DMSO), dimethylformide (DMF)) or a mixture of two or more thereof. The antibody or variant thereof according to the present invention may be of any species or origin. It may bind to any epitope(s) comprised by its molecular target structure (e.g., linear epitope(s), structural epitope(s), primary epitope(s), secondary epitope(s), i.e., such of Vim3). Preferably, the antibody or variant thereof may recognize the naturally folded molecular target structure or a domain or fragment thereof (e.g., Vim3 in the environment of blood serum). The antibody or variant thereof may be of any origin an antibody may be obtained from such as, e.g., natural origin, a gene technologic origin and/or a synthetic origin. Optionally, the antibody may also be commercially available. The person skilled in the art will understand that the antibody may further comprise one or more posttranscriptional modification(s) and/or may be conjugated to one or more further structures such as label moieties or cell-penetrating peptides (CPPs). Optionally, the antibody or antibody fragment may be added to a support, particularly a solid support such as an array, bead (e.g. glass or magnetic), a fiber, a film etc. The skilled person will be able to adapt the antibody of the present invention and a further component to the intended use by choosing a suitable further component.

In a particularly preferred embodiment, the present invention relates to a method for detecting a malignant carcinoma in an individual, said method conducted in vitro comprising the following steps:

(i) providing blood serum S obtained from the individual; and
(ii) determining the level of Vim3 polypeptide in the blood serum S,
wherein an increased level of Vim3 polypeptide in the blood serum S indicates the presence of a malignant carcinoma in the individual.

In another particularly preferred embodiment, the present invention relates to a method for detecting a malignant carcinoma in an individual, said method conducted in vitro comprising the following steps:
(i) providing blood serum S obtained from the individual;
(ii) determining the level of Vim3 polypeptide in the blood serum S; and
(iii) comparing the Vim3 level determined in step (ii) with a predetermined reference value R1 indicating the borderline between blood serum indicating the presence of a malignant tumor and a blood serum of the same species indicating the absence of a malignant tumor,
   wherein an Vim3 level determined in the blood serum S that is higher than R1 indicates the presence of a malignant tumor in the individual, and
   wherein the Vim3 level is related to the total polypeptide content comprised in the respective sample.

In another particularly preferred embodiment, the present invention relates to a method for detecting a malignant carcinoma in an individual, said method conducted in vitro comprising the following steps:
(i) providing blood serum S obtained from the individual;
(ii) determining the level of Vim3 polypeptide in the blood serum S; and comparing the Vim3 level determined in step (ii) with a Vim3 level determined in a control sample C obtained from one or more control individuals of the same species free of a malignant tumor,
   wherein an Vim3 level determined in the blood serum S that is at least 10% higher than the Vim3 level of C indicates the presence of a malignant tumor in the individual, and
   wherein the Vim3 level is related to the total polypeptide content comprised in the respective sample.

In an alternative preferred embodiment, the step (ii) of determining the Vim3 level is determining the level of Vim3 messenger RNA. In a more preferred embodiment, the step (ii) of determining the Vim3 level is determining the level of Vim3 messenger RNA by means of conducting at least one step selected from the group consisting of polymerase chain reaction (PCR), real time PCR (RT-PCR), by in situ hybridization, gel electrophoresis, Southern Blot, and combinations of two or more thereof.

In this context, step (ii) comprises determining the respective mRNA. The latter may be conducted equivalent to the process as described by von Brandenstein et al. in 2012 for PKC-alpha (cf., The American Journal of Pathology, 2012, 180:1787-1797). Vim3 mRNA may be also detected as described in WO 2014/154686, in particular by means of PCR techniques based on the primers described therein.

In order to improve comparability of different blood serums S with another, in other words to normalize the determined results, the level of Vim3 mRNA determined in a sample is preferably related to a reference value such as the sample volume, the total nucleotide or mRNA content comprised in same sample (e.g., the blood serum S or a control sample) or the amount of an intrinsic marker (e.g., albumin, transferrin and/or beta-actin polypeptide or albumin, transferrin and/or beta-actin mRNA) of known concentration naturally contained in the same sample (e.g., the blood serum S or a control sample). If related to the sample volume, the level indicates the concentration, i.e., the respective mRNA per volume (e.g, mass of Vim3 mRNA per volume of the blood serum S [ng/ml]). If related to the total nucleotide or mRNA content, a relative ratio may be provided (e.g, mass of Vim3 mRNA per mass of total nucleotide or mRNA content of the blood serum S [pg/ng]). If related to the content of an intrinsic marker, a relative ratio may be provided (e.g, mass of Vim3 mRNA per mass of transferrin [pg/pg] or the mass of Vim3 mRNA per mass of beta-actin [pg/pg], or Vim3 mRNA per mass of albumin mRNA [pg/pg], or Vim3 mRNA per mass of transferrin mRNA [pg/pg] or the mass of Vim3 mRNA per mass of beta-actin mRNA [pg/pg] or the mass of Vim3 mRNA per mass of albumin mRNA [pg/ng].

Highly preferably, the step of determining the Vim3 level is determining the level of Vim3 mRNA in relation to the total polypeptide, the total nucleotide, or the total mRNA content comprised in the respective sample.

It will be understood that the present method may be optionally combined with any other method indicating the presence or absence of a tumor, including metastasis in the individual of interest. Exemplarily, the method of the present invention may be combined with detecting the level of prostate-specific antigen (PSA) (also designated as gamma-seminoproteine and kallikrein-3 (KLK3)). Furthermore, the method of the present invention, in particular when indicating the presence of a tumor (in particular such including metastases) may optionally be followed by any method usable for medical imaging such exemplarily a method selected from those based on radiography, magnetic resonance imaging (MRI), nuclear medicine, ultrasound, elastography, tactile imaging, photoacoustic imaging, tomography, echocardiography, functional near-infrared spectroscopy, magnetic particle imaging, and combinations of two or more thereof.

Means for determining the Vim3 level in blood serum S may be any means suitable for this purpose. It is referred to the method of the present invention as laid out herein. In particular, such means may comprise Vim3-specific antibodies or variants or fragments thereof, and optionally secondary antibodies. Such antibodies or variants or fragments thereof, and optionally secondary antibodies may optionally be immobilized and/or labelled. This is described in more detail below. Preferably, such means are suitable for detecting the Vim3 polypeptide. Alternatively or additionally, such means may comprise primers (typically a forward and a reverse primer) for amplifying the Vim3 mRNA.

In a particularly preferred embodiment, the means for determining the Vim3 level) form part of a dipstick. This is described in more detail below.

A kit according to the present invention may include a solid phase and a capture agent affixed to the solid phase, wherein the capture agent is an antibody specific for the analysis of a blood serum S (e.g., at least one Vim3-specific antibody). The solid phase may comprise a material such as a magnetic or paramagnetic particle including a microparticle, a bead, a test tube, a microtiter plate, a cuvette, a membrane, a scaffolding molecule, a quartz crystal, a film, a filter paper, a dipstick a disc or a chip.

Furthermore, a kit according to the present invention may preferably further comprise user instructions for carrying out the method of the present invention. Instructions included in kits of the invention may be affixed to packaging material or may be included as a package insert. While the instructions are typically written or printed materials they are not limited to such. Any medium capable of storing such instructions and communicating them to an end user is contemplated by this invention. Such media include, for example, computer media including, but are not limited to, electronic storage media (e.g., magnetic discs, tapes, cartridges, chips), optical media (e.g., CD ROM), and the like.

As indicated above, the method of the present invention can also be conducted by means of a dipstick analysis (lateral flow analysis).

Accordingly, a still further aspect of the present invention refers to a dipstick usable for the method of the present invention comprising, placed in the direction of flow of the blood serum S, on a carrier that is suitable for soaking the blood serum S, the following:
(0) an edge or segment suitable for soaking the blood serum S;
(1) optionally a stripe (1) comprising labeled Vim3-specific antibodies or antibody fragments which are not immobilized and freely movable when the blood serum S passes through this stripe (1);
(2) a stripe (2) comprising immobilized unlabeled vimentin-specific, in particular Vim3-specific, antibodies or antibody fragments; and
(3) optionally a stripe (3) of immobilized unlabeled antibodies or antibody fragments specifically binding the labeled Vim3-specific antibodies or antibody fragments of stripe (1).

Accordingly, a dipstick according to the present invention (preferably usable for the method of or the use of the present invention) comprise at least, placed in the direction of flow of the blood serum, on a carrier that is suitable for soaking the blood serum S, the following:
(0) an edge or segment suitable for soaking the blood serum S; and
(2) a stripe (2) comprising immobilized unlabeled vimentin-specific, in particular Vim3-specific, antibodies or antibody fragments.

As used herein, the terms “dipstick”, “dip-stick”, “test strip”, “control strip”, “diagnostic/medical dipstick” may be understood interchangeably in the broadest sense as any device that is usable to test a blood serum S in the context of the present invention (according to the lateral flow technique). In the context of the dipstick, the blood serum S is typically liquid, semi-liquid or liquefied so that it can be soaked by a carrier of the dipstick. Typically, the blood serum S comprises an aqueous liquid. Exemplarily, the blood serum S usable by the dipstick may be blood serum from an individual.

In particular if the dipstick lacks stripe (1), the blood serum S is preferably premixed with a labeled Vim3-specific antibody or antibody fragment or fragment thereof. The volume and molar ratios will be adapted accordingly in order to optimize binding efficiency.

The volume of the blood serum S (optionally diluted and/or premixed with a labeled Vim3-specific antibody or antibody fragment) added to the dipstick will be adapted to the size and material of the dipstick. Typical volumes for adding to a segment suitable for soaking the blood serum S are in the range of from 10 to 1000 μl, preferably 50 to 500 μl, in particular 75 to 300 μl, exemplarily (approximately 200 μl). Exemplarily, the carrier may be a (hydro) gel or a piece of paper board, and may be optionally film laminated. Typically, the dipstick will be stored in dry state and is moistened by the blood serum S. When conducting the method of the present invention by means of the dipstick, the edge or segment suitable for soaking the blood serum S(0) may be contacted with the blood serum S. This is preferably conducted long enough to enable the sample liquid to be soaked in the carrier of the dipstick. The other parts of the dipstick are preferably not directly contacted with the blood serum S.

It is preferably enabled that the blood serum S flows through the carrier of the dipstick at least until the stripes (1) (if present) and (2) and optionally (3) have been passed by the blood serum S or parts thereof.

According to a preferred embodiment, the blood serum S is of a first species and the antibodies or antibody fragments of each of stripe (1) (if present) or the antibodies or fragments used for premixing with the blood serum S (in particular if stripe (1) is not present) on the one hand and (2) and optionally (3) of the other hand are each of different species.

In a preferred embodiment, the immobilized unlabeled antibodies or antibody fragments of stripe (3) specifically bind to the Fc fragment of the labeled Vim3-specific antibodies or antibody fragments of stripe (1) (if present) or premixed with the optionally diluted blood serum (in particular if stripe (1) is not present). Exemplarily, the Vim3-specific antibodies or antibody fragments which are not immobilized are (preferably monoclonal) rabbit antibodies. Then, the immobilized antibodies of stripe (3) may be (preferably monoclonal) antibodies directed against the Fc part of the antibodies provided in stripe (1) or premixed with the blood serum sample and optionally one or more buffers (in particular if stripe (1) is not present).

The label may be a fluorescence label, a visible dye label or, particularly preferably, a (colloidal) gold label. Such (colloidal) gold may be added to an antibody or antibody fragment bay any means, exemplarily by means of a GOLD Conjugation Kit.

When a Vim3-containing blood serum S sample of an individual bearing a malignant tumor, in particular a malignant carcinoma, is added to the dipstick, upon flowing through the dipstick, the labeled Vim3-specific antibodies may bind to Vim3 in the blood serum S, thereby forming a Vim3/antibody conjugate. This conjugate will then binding to the unlabeled Vim3-specific antibodies of stripe.

When a sample lacking Vim3 of a healthy individual is added to the dipstick, upon flowing through the dipstick, the labeled Vim3-specific antibodies will not form a Vim3/antibody conjugate. Therefore, the polypeptides comprised in the blood serum S will then pass by the stripe (2) without being bound and will pass through the dipstick until the stripe (3). In such dipstick, the ratio between signal intensity of the label in stripe (2) and (3) may indicate the presence or absence of malignant tumor in the individual the blood serum S has been obtained from. A higher (2):(3) ratio indicates higher probability of the presence of a malignant tumor in the individual, whereas a lower (2):(3) ratio indicates lower probability of the presence of a malignant tumor in the individual in the sense of the method of the present invention laid out above.

The premixing of the labeled Vim3-specific antibodies or antibody fragments may be followed by an incubation to allow and optimize binding of the Vim3-specific antibodies or antibody fragments to its molecular target Vim3. This may exemplarily be performed by incubating for 10 to 60 min at a temperature of from 2 to 25° C.

Alternatively or additionally, a dipstick according to the present invention may be may be prepared according to Preechakasedkit et al., 2012, Biosens Bioelectron 31(1):562-566; Tao et al., 2014, Lett Appl Microbiol 59(2):247-251 or Wang et al., 2010, J Virol Methods 2010, 170(1-2):80-85.

It will be understood that the alteration of the cells also occurs at the cellular level. Surprisingly, it was found that in solid tissue samples T, Vim3 polypeptide accumulates in cell nuclei in cancerous cells.

The present invention further relates to a method for detecting a malignant tumor in an individual, said method conducted in vitro comprising determining the intracellular localization of Vim3 polypeptide in individual's solid tissue sample T. Herein, preferably, an accumulation of Vim3 polypeptide in the cell nuclei in the solid tissue sample T indicates the presence of a malignant tumor in the individual.

Accordingly, a further aspect of the present invention relates to a method for detecting a malignant tumor in an individual, said method conducted in vitro comprising the following steps:
(i) providing a solid tissue sample T obtained from the individual; and
(ii) determining the intracellular localization of Vim3 polypeptide in the solid tissue sample T,
   wherein an accumulation of Vim3 polypeptide in the cell nuclei in the solid tissue sample T indicates the presence of a malignant tumor in the individual.

It will be understood that the specifications made in the context of the methodical steps of detecting Vim3 in blood serum S as described above apply mutatis mutandis to the methodical steps of determining the intracellular localization of

Vim3 polypeptide in the solid tissue sample T as far as scientifically appropriate. In other words, Vim3 accumulations in the cell nuclei may be used as a marker for malignancy. Vim3 in the cytosol rather indicates benignancy. It has been found that this finding is less prominent in a malignant renal tumor, in particular in a malignant renal cell carcinoma (RCC) (compared to a tumor, in particular carcinoma, of another tissue). This may be due to the low overall Vim3 level in renal cells, including healthy renal cells.

In a preferred embodiment, in the context of this method based on the accumulation of Vim3 polypeptide in the cell nuclei the malignant tumor is not a malignant renal tumor (i.e., is a non-renal malignant tumor). In a preferred embodiment in this context, the malignant tumor is preferably not a renal carcinoma (i.e., is a non-renal carcinoma), in particular not renal cell carcinoma (RCC).

In another preferred embodiment, the malignant tumor is a carcinoma. In a more preferred embodiment, the malignant tumor is a carcinoma except a renal carcinoma, in particular renal cell carcinoma (RCC). In a more preferred embodiment, the malignant tumor is a malignant carcinoma selected from the group consisting of prostate carcinoma, urothelial carcinoma, bladder carcinoma, transitional cell carcinoma, mucoepidermiod carcinoma, mammacarcinoma, small-cell carcinoma, myoepithelial carcinoma, adenocarcinoma, gastric signet ring cell carcinoma, and esophageal carcinoma. Even more preferably, malignant carcinoma is selected from the group consisting of breast adenocarcinoma, small-cell lung adenocarcinoma, prostate adenocarcinoma, mucoepidermoid carcinoma of the salivary glands, and urothelial carcinoma of the bladder.

In a preferred embodiment, the solid tissue sample T is a sample from a tissue that is of risk being or comprising a tumor (i.e., parent tumor or one or more metastases).

In another particularly preferred embodiment, the present invention relates to a method for detecting a malignant carcinoma in an individual, said method conducted in vitro comprising the following steps:
(i) providing a solid tissue sample T from a tissue that is of risk being or comprising a tumor obtained from the individual; and
(ii) determining the intracellular localization of Vim3 polypeptide in the solid tissue sample T,
   wherein an accumulation of Vim3 polypeptide in the cell nuclei in the solid tissue sample T indicates the presence of a malignant carcinoma in the individual.

In a preferred embodiment, the step (ii) of determining the intracellular localization of Vim3 polypeptide in the solid tissue sample T is performed by means of microscopy. Alternatively or additionally, it may also be performed by determining the colocalization with cell nucleus staining.

In a more preferred embodiment, the step (ii) of determining the intracellular localization of Vim3 polypeptide in the solid tissue sample T is performed by means of microscopy, wherein the cell nucleus staining are stained by a dye selectively staining the a cell nucleus (e.g. DAPI, hematoxylin) and concomitantly by a staining Vim3, and subsequently colocalization of the Vim3 staining with cell nucleus staining is determined. The cells may also be stained by an hematoxylin and eosin stain (HE stain).

In a preferred embodiment, the solid tissue sample T may be a fixed sample. In a more preferred embodiment, the solid tissue sample T may be a fixed sample embedded in paraffin and cut unto sections (e.g. in the range of 1 to 100 μm, preferably 1.5 to 50 μm, more preferably 2 to 20 μm, even more preferably 2.5 to 10 μm, even more preferably 2.5 to 5 μm in particular (approximately) 3 μm thickness). In a highly preferred embodiment, if paraffin is used to prepare sections, the paraffin is washed out by means of xylene followed by washing steps with ethanol and ethanol/water mixtures with increasing contents of water, and finally followed by one or more washing steps with distilled water.

In a preferred embodiment, the sections (slides) are incubated with a serum blocker (e.g., for 30 minutes) or with a protein solution (e.g., 5% PBS milk) in order to avoid nonspecific binding.

The detection of step (ii) may be conducted via microscopy, in particular fluorescence-based microscopy. It may also be conducted via laser-scanning microscopy (LSM). Alternatively, the sample may also be stained by chromatic dyes (e.g., organic, inorganic, metal based beads, etc.) visible in bright field microscope.

Vim3 may be stained with a fluorescent-labelled Vim3-specific antibody or an unlabeled Vim3-specific first antibody (e.g., Vim3 antibody, EZBiolab, Inc. Carmel, USA) that is bound via a labelled secondary antibody binding to the first antibody. Further specific antibodies or an unlabeled specific first antibody (e.g., Vimentin V9, Santa Cruz, Heidelberg, Germany, AMACR and CD117, Dako, Hamburg, Germany) may likewise be bound via a labelled secondary antibody binding to the first antibody.

Optionally, the Vim3-specific antibodies may be added in protein-containing solution such as a PBS milk solution. In a preferred embodiment, between binding of the first and the second antibody, the section of the solid tissue sample T is washed in order to remove the excess of the first antibody (e.g., by mixtures of phosphate buffered saline (PBS) and an emulsifier like Tween 20 (PBS-Tween 20)). Then the secondary antibody directed to the Fc part of the first antibody may be added (e.g., anti-rabbit antibody (Santa Cruz, Heidelberg, Germany, that may be FITC-labeled). Again, preferably, the solid tissue sample T may be washed in order to remove the excess of the second antibody (e.g., by mixtures of PBS and an emulsifier like Tween 20 (PBS-Tween 20)). This may optionally be followed by incubating with ethanol and/or methanol. The solid tissue sample T may be counterstained with an HE stain and/or with a DAPI stain, and may optionally be cover slipped.

The person skilled in the art is well-aware of such antibody systems. Examples are described more in detail herein, such as in the context of the methodical steps of detecting Vim3 in blood serum S above and in the example section below. More specific examples are provided below in eth Example section.

In a preferred embodiment, the method additionally comprises the step of:
(iiia) determining the intracellular localization of Atg7 polypeptide in the solid tissue sample T,
   wherein colocalization of Vim3 and Atg7 polypeptides, in particular in the form of one or more accumulations, in the cell nuclei in the solid tissue sample T indicates the presence of a malignant tumor in the individual.

In another preferred embodiment, the method additionally comprises the step of:
(iiib) determining the intracellular localization of p53 polypeptide in the solid tissue sample T,
   wherein colocalization of Vim3 and p53 polypeptides, in particular in the form of one or more accumulations, in the cell nuclei in the solid tissue sample T indicates the presence of a malignant tumor in the individual.

In a more preferred embodiment, the method additionally comprises the steps of:
(iiia) determining the intracellular localization of Atg7 polypeptide in the solid tissue sample T; and
(iiib) determining the intracellular localization of p53 polypeptide in the solid tissue sample T,
   wherein colocalization of Vim3, Atg7 and p53 polypeptides, in particular in the form of one or more accumulations, in the cell nuclei in the solid tissue sample T indicates the presence of a malignant tumor in the individual.

As indicated above, it has been surprisingly found that in malignant tumor cells particularly large amounts of Vim3 forms complexes with Atg7 and/or p53 that preferably tend to migrate in the cell nuclei.

The present invention further relates to a method for detecting a malignant tumor in an individual, said method conducted in vitro comprising determining complexes comprising Vim3 and at least one further protein selected from Atg7, p53, and a combination of both in a solid tissue sample T, that is preferably a sample from a tissue that is of risk being or comprising a tumor, obtained from the individual. Herein, preferably, the presence of complexes in the in the solid tissue sample T, in particular in the cell nuclei in the solid tissue sample T, indicates the presence of a malignant tumor in the individual.

Accordingly, a further aspect of the present invention relates to a method for detecting a malignant tumor in an individual, said method conducted in vitro comprising the following steps:
(i) providing a solid tissue sample T, that is preferably a sample from a tissue that is of risk being or comprising a tumor, obtained from the individual; and
(ii) determining complexes comprising Vim3 and at least one further protein selected from Atg7, p53, and a combination of both,
   wherein the presence of complexes in the in the solid tissue sample T, in particular in the cell nuclei in the solid tissue sample T, indicates the presence of a malignant tumor in the individual.

It will be understood that the specifications made in the context of the methodical steps of detecting Vim3 in blood serum S as described above as well as in the solid tissue sample T as described above apply mutatis mutandis to the methodical steps of determining complexes as far as scientifically appropriate.

In a preferred embodiment, step (ii) is determining complexes comprising Vim3, Atg7 and p53 (Vim3/Atg7/p53 complexes).

It will be understood that in this context also Atg7 and/or p53 may be stained by any means. Examples for antibody types usable in this context are laid out above.

Exemplarily, Atg7 may be stained with a fluorescent-labelled Atg7-specific antibody or an unlabeled Atg7-specific first antibody that is bound via a labelled secondary antibody binding to the first antibody. The person skilled in the art is well-aware of such antibody systems. Examples are described more in detail herein, such as in the context of the methodical steps of detecting Atg7 in blood serum S above and in the example section below.

Exemplarily, p53 may be stained with a fluorescent-labelled p53-specific antibody or an unlabeled p53-specific first antibody that is bound via a labelled secondary antibody binding to the first antibody. The person skilled in the art is well-aware of such antibody systems. Examples are described more in detail herein, such as in the context of the methodical steps of detecting p53 in blood serum S above and in the example section below.

In a preferred embodiment, the individual (potentially) having one or more malignant tumors, has been treated with an anticancer agent, in particular tamoxifen (before). Such anticancer agent such as tamoxifen, may lead to an increase of endothelin (ET-1) levels that may lead to increased levels of Vim3. The molecular background is described in more detail in von Brandenstein et al, 2018, Journal of Cancer Therapy 9:438-463 and von Brandenstein et al., 2018, Oncotarget 9:32855-32867.

In a preferred embodiment, in addition to determining the Vimentin variant 3 (Vim3) level in the blood serum S, also the endothelin (ET-1) level in the blood serum S may be determined, wherein an increased ET-1 level in the blood serum S may indicate the presence of a malignant tumor in the individual. Thus, ET-1 may serve as a co-marker of Vim3.

In a preferred embodiment, the method of the present invention comprises a further step of treating the individual bearing (and optionally suffering from) at least one malignant tumor.

As used herein, the term “treating” may be understood in the broadest sense. The treating step as described herein may be the sole treatment of the individual or may be combined with one or more other treatment(s) of the individual.

In a preferred embodiment, treating the malignant tumor that is preferably identified by a method of the present invention is or comprises administering the individual with an amount of an antineoplastic agent sufficient for treating the type of malignant tumor identified in the preceding step.

Accordingly, in a preferred embodiment, the method of the present invention comprises a further step of administering the individual bearing (and optionally suffering from) a malignant tumor with an amount of an antineoplastic agent sufficient for treating said malignant tumor. In a highly preferred embodiment, the malignant tumor is endothelin B receptor negative.

In other words, a further aspect of the present invention relates to an antineoplastic agent for use in a method for treating an individual bearing a malignant tumor, wherein the presence of the malignant tumor in the individual has previously been detected in the individual by means of a method of the present invention.

In other words, a further aspect of the present invention relates to an antineoplastic agent for use in a method for treating an individual bearing a malignant tumor, wherein the presence of the malignant tumor in the individual has previously been detected in the individual by means of a method of the present invention.

As used herein, the terms “antineoplastic agent”, “anticancer agent”, “antineoplastic drug”, “anticancer drug”, “anticancer compound”, “antineoplastic compound” and equivalents thereof may be understood interchangeably in the broadest sense as any agent that is suitable for treating a malignant tumor (i.e., cancer). Exemplarily, such antineoplastic agent may be selected from the group consisting of chemotherapeutics, hormones and analogue thereof and other antineoplastic agent.

Exemplarily, such antineoplastic agent may be selected from the group consisting of platins (e.g., cisplatin, carboplatin, oxaliplatin), anti-metabolites (e.g., azathioprine, 6-mercaptopurine, mercaptopurine, 5-fluorouracil, pyrimidines, thioguanine, fludarabine, floxuridine, cytosine arabinoside (cytarabine), pemetrexed, raltitrexed, pralatrexate, methotrexate), further alkylating agents (e.g., chlorambucil, Ifosfamide mechlorethamine, cyclophosphamide), statins (e.g., cerivastatin, simvastatin, lovastatin, somatostatin, fluvastatin, nystatin, rosuvastatin, atorvastatin, pravastatin, pitavastatin, pentostatin), terpenoids and plant alkaloids (e.g., vinca alkaloids (vincristine, vinblastine, vinorelbine, vindesine), taxanes (e.g., paclitaxel), cytoxan), topoisomerase inhibitors (e.g., camptothecins: irinotecan, topotecan, etoposide, etoposide phosphate, teniposide), melphalan, other antineoplastica (e.g., doxorubicin (adriamycin), doxorubicin lipo, epirubicin, bleomycin)), actinomycin D, aminoglutethimide, amsacrine, anastrozole, antagonists of purine and pyrimidine bases, anthracyclines, aromatase inhibitors, asparaginase, antiestrogens, bexarotene, buserelin, busulfan, camptothecin derivatives, capecitabine, carmustine, cladribine, cytarabine, cytosine arabinoside, alkylating cytostatics, dacarbazine, daunorubicin, docetaxel, epirubicin, estramustine, etoposide, exemestane, fludarabine, fluorouracil, folic acid antagonists, formestane, gemcitabine, glucocorticoids, goserelin, hormones and hormone antagonists, hycamtin, hydroxyurea, idarubicin, irinotecan, letrozole, leuprorelin, lomustine, mercaptopurine, miltefosine, mitomycins, mitosis inhibitors, mitoxantrone, nimustine, procarbazine, tamoxifen, temozolomide, teniposide, testolactone, thiotepa, topoisomerase inhibitors, treosulfan, tretinoin, triptorelin, trofosfamide, cytostatically active antibiotics, everolimus, pimecrolimus, tacrolimus, azithromycin, spiramycin, sirolimus (rapamycin), roxithromycin, ascomycin, bafilomycin, erythromycin, midecamycin, josamycin, concancamycin, clarithromycin, troleandomycin, folimycin, tobramycin, mutamycin, dactinomycin, dactinomycin, rebeccamycin, 4-hydroxyoxycyclophosphamide, bendamustine, thymosin α-1, aclarubicin, fludarabine-5′-dihydrogen phosphate, hydroxycarbamide, aldesleukin, pegaspargase, cepharanthine, epothilone A and B, azathioprine, mycophenolate mofetil, c-myc antisense, b-myc antisense, betulinic acid, camptothecin, melanocyte stimulating hormone (α-MSH), activated protein C, IL-1β inhibitor, fumaric acid and esters thereof, dermicidin, calcipotriol, taclacitol, lapachol, β-lapachone, podophyllotoxin, betulin, podophyllic acid 2-ethyl hydrazide, sagramostim, (rhuGM-CSF), peginterferon α-2b, lenograstim (r-HuG-CSF), filgrastim, macrogol, cephalomannine, selectin (cytokine antagonist), CETP inhibitor, cadherins, cytokinin inhibitors, agrostistachin, 17-hydroxyagrostistachin, ovatodiolids, 4,7-oxycycloanisomelic acid, baccharinoids B1, B2, B3 and B7, tubeimoside, bruceanol A, B and C, bruceantinoside C, yadanziosides N and P, isodeoxyelephantopin, tomenphantopin A and B, coronarin A, B, C and D, ursolic acid, COX inhibitor (e.g., COX-2 and/or COX-3 inhibitor), angiopeptin, ciprofloxacin, fluroblastin, bFGF antagonists, probucol, prostaglandins, 1,11-dimethoxyeanthin-6-one, 1-hydroxy-11-methoxycanthin-6-one, scopoletin, colchicine, NO donors, pentaerythrityl tetranitrate, sydnonimines, S-nitroso derivatives, staurosporine, β-estradiol, α-estradiol, estriol, estrone, ethinyl estradiol, fosfestrol, medroxyprogesterone, estradiol cypionates, cudraisoflavone A, curcumin, dihydronitidine, nitidine chloride, 12-beta-hydroxypregnadiene-3,20-dione bilobol, ginkgol, ginkgolic acid, helenalin, indicine, indicine-N-oxide, lasiocarpine, inotodiol, glycoside 1a, justicidin A and B, larreatin, malloterin, mallotochromanol, isobutyrylmallotochromanol, marchantin A, maytansine, lycoridicin, margetine, pancratistatin, liriodenine, bisparthenolidine, oxoushinsunine, aristolactam-All, estradiot benzoates, tranilast, kamebakaurin, verapamil, ciclosporin A, paclitaxel and derivatives thereof such as 6-α-hydroxy paclitaxel, baccatin, taxotere, mofebutazone, acemetacin, diclofenac, lonazolac, dapsone, o-carbamoyl-phenoxy-acetic acid, lidocaine, ketoprofen, mefenamic acid, piroxicam, meloxicam, chloroquine phosphate, penicillamine, hydroxychloroquine, auranofin, sodium aurothiomalate, oxaceprol, celecoxib, β-sitosterol, ademetionine, myrtecaine, polidocanol, nonivamide, levomenthol, benzocaine, aescin, elipticine, D-24851 (Calbiochem), colcemid, cytochalasin A-E, indanocine, nocodazole, bacitracin, vitronectin receptor antagonists, azelastine, free nucleic acids, nucleic acids incorporated into virus transmitters, DNA and RNA fragments, plasminogen activator inhibitor-1, plasminogen activator inhibitor-2, antisense oligonucleotide, VEGF inhibitors, IGF-1, active agents from the group of antibiotics such as cefadroxil, cefazolin, cefaclor, cefoxitin, gentamicin, penicillins, dicloxacillin, oxacillin, sulfonamides, metronidazole, antithrombotics, argatroban, aspirin, abciximab, synthetic antithrombin, bivalirudin, coumadin, enoxaparin, GpIIb/IIIa platelet membrane receptor, antibodies to factor Xa inhibitor, heparin, hirudin, r-hirudin, PPACK, protamine, prourokinase, streptokinase, warfarin, urokinase, vasodilators, dipyramidole, trapidil, nitroprussides, PDGF antagonists, triazolopyrimidine, seramin, ACE inhibitors, captopril, cilazapril, lisinopril, enalapril, losartan, thioprotease inhibitors, prostacyclin, vapiprost, interferon α, β and γ, histamine antagonists, serotonin blockers, apoptosis inhibitors, apoptosis regulators, NF-kB, Bcl-xL antisense oligonucleotides, halofuginone, nifedipine, tocopherol, molsidomine, tea polyphenols, epicatechin gallate, epigallocatechin gallate, boswellic acids and derivatives thereof, leflunomide, anakinra, etanercept, sulfasalazine, tetracycline, triamcinolone, procainimide, retinoic acid, quinidine, disopyramide, flecainide, propafenone, sotalol, amiodarone, natural and synthetically obtained steroids such as withaferin A, bryophyllin A, inotodiol, maquiroside A, mansonine, strebloside, hydrocortisone, betamethasone, dexamethasone, fenoprofen, ibuprofen, indomethacin, naproxen, phenylbutazone, acyclovir, ganciclovir, zidovudine, antimycotics, clotrimazole, flucytosine, griseofulvin, ketoconazole, miconazole, terbinafine, chloroquine, mefloquine, quinine, natural terpenoids, hippocaesculin, barringtogenol-C₂₁-angelate 14-dehydroagrostistachin, agroskerin, hyptatic acid A, zeorin, strychnophylline, usambarine, usambarensine, daphnoretin, lariciresinol, methoxylariciresinol, syringaresinol, umbelliferone, afromoson, acetylvismione B, desacetylvismione A, vismione A and B, iso-iridogermanal, maytenfoliol, effusantin A, excisanin A and B, longikaurin B, sculponeatin C, kamebaunin, leukamenin A and B, 13,18-dehydro-6-alpha-senecioyloxychaparrine, taxamairin A and B, regenilol, triptolide, cymarin, apocymarin, aristolochic acid, anopterin, hydroxyanopterin, anemonin, protoanemonin, berberine, cheliburin chloride, cicutoxin, sinococuline, combrestatin A and B, periplocoside A, ghalakinoside, deoxypsorospermin, psychorubin, ricin A, sanguinarine, manwu wheat acid, methylsorbifolin, chromones of spathelia, stizophyllin, akagerine, dihydrousambaraensine, hydroxyusambarine, strychnopentamine, a pharmaceutically acceptable salt of any thereof, and a combination of two or more thereof or two or more pharmaceutically acceptable salts thereof.

An antineoplastic agent may also be an agent suitible for immunotherapy of malignant tumors. An agent suitible for immunotherapy of malignant tumors may be understood in the broadest sense as any agent suitible to stimulate the immune system to treat malignant tumors. It may be active, passive or a mixture of both (hybrid). In this context, immunotherapy may base on the detectability of neoplasm-associated antigens (often also designated as tumour-associated antigens (TAAs)). Active immunotherapy may direct the immune system to attack neoplastic cells by targeting neoplasm-associated antigens. Passive immunotherapies may enhance existing antineoplastic responses and include the use of antibodies or fragments or variants thereof, immune cells (e.g., lymphocytes (e.g., T-lymphocytes, B-lymphocytes), natural killer cells, lymphokine-activated killer cells, cytokine-activated killer cells, cytotoxic T cells and dendritic cells) and/or cytokines, in particular (optionally humanized) monoclonal antibodies or fragments thereof. Depending on the individual setup, such antibodies or fragments or variants thereof, immune cells and/or cytokines may lead to antibody-dependent cell-mediated cytotoxicity, may activate the complement system, and/or may prevent a receptor from interacting with its ligand. Thereby, in some setups, the targeted cell may be triggered into apoptosis. Examples for antibodies usable in the context of immune therapy include alemtuzumab, ipilimumab, nivolumab, ofatumumab and rituximab. Antibodies or fragments or variants thereof may optionally also be conjugated (e.g., by a radioactive ion). Additionally or alternatively, also dendritic cell therapy may be used. Additionally or alternatively, also cytokines, keyhole limpet hemocyanin, Freund's adjuvant, Bacillus Calmette-Guérin (BCG) vaccine and/or peginterferon alfa-2a may be used. Alternatively or additionally, also an antineoplastic vaccine may be used such as, e.g., a vaccine made of neoplastic tissue or an artificial vaccine (e.g., polypeptide-based, polynucleotide-based, glycoside-based, etc.). The person skilled in the art will be aware of several further agents suitible for immunotherapy of malignant tumors usable in the context of the present invention.

In a highly preferred embodiment, the antineoplastic agent for use of the present invention, wherein said antineoplastic agent is selected from the group consisting of withaferin A and Vim3 siRNA.

In a particularly preferred embodiment, the antineoplastic agent for use of the present invention is withaferin (A).

In a preferred embodiment, the antineoplastic agent for use of the present invention is used in combination with tamoxifen. In a preferred embodiment, withaferin A and/or Vim3 siRNA for use of the present invention is/are used in combination with tamoxifen. In particular, a combination of withaferin A and tamoxifen are used in a method for treating or preventing a malignant tumor.

Accordingly, another aspect of the present invention also relates to an antineoplastic agent comprising

(A) withaferin A and/or Vim3 siRNA; and
(B) tamoxifen
for use in a method for treating or preventing a malignant tumor in an individual.

It will be understood that the definitions and preferred embodiment as defined in the context of the present invention mutatis mutandis also apply to this aspect of treating or preventing a malignant tumor.

In other words, the present invention also relates to a method for treating or preventing a malignant tumor in an individual, said method comprising administering a sufficient amount of

(A) a sufficient amount of withaferin A and/or Vim3 siRNA; and
(B) a sufficient amount of tamoxifen
to said individual.

Alternatively or additionally, treating may also be or comprise surgical means of excising the neoplastic tissue, in particular a malignant tumor tissue, from the individual's body.

Alternatively or additionally, treating may also be or comprise radiation therapy suitible for treating one or more malignant tumors (i.e., cancer), in particular malignant tumor tissue. Radiation therapy may exemplarily be or comprise irradiation with x-ray radiation, ultraviolet (UV) radiation (e.g., UV-A, UV-B, and/or UV-C radiation), alpha radiation, beta radiation, gamma radiation, or cosmic radiation. Radiation therapy may also include, but may not be limited to Intensity-Modulated Radiation Therapy (IMRT), 3-Dimensional Conformal Radiotherapy (3DCRT), Stereotactic body radiation therapy (SBRT), Stereotactic radiosurgery (SRS), image-guided radiation therapy (IGRT), Particle Therapy (e.g, proton therapy), Brachytherapy, Radioisotope Therapy (RIT) (e.g., with iodine-131, lutetium-177, strontium-89 and samarium (¹⁵³Sm) lexidronam and/or yttrium-90).

In a preferred embodiment, the invention relates to a method for treating an individual bearing (and optionally suffering from) a malignant tumor, said method comprising:

the steps of the method for detecting a malignant tumor in an individual according to the invention, and
the step of treating the malignant tumor identified in the preceding step.

A still further aspect of the present invention relates to an antineoplastic agent that is or comprises a Vim3 inhibitor for use in a method for treating an individual bearing a malignant tumor, wherein said malignant tumor is endothelin B receptor negative.

In other words, the present invention relates to method for treating an individual bearing a malignant tumor, comprising administering the individual with an amount of a Vim3 inhibitor sufficient for treatment, wherein said malignant tumor is endothelin B receptor negative.

In a preferred embodiment, the antineoplastic agent for use of the present invention, wherein said antineoplastic agent is selected from the group consisting of withaferin A and Vim3 siRNA.

The invention is not limited to the particular methodology, protocols, and reagents described herein because they may vary. Further, the terminology used herein is for the purpose of describing particular embodiments only and is not intended to limit the scope of the present invention. As used herein and in the appended claims, the singular forms “a”, “an”, and “the” include plural reference unless the context clearly dictates otherwise. Similarly, the words “comprise”, “contain”, “include” and “encompass” are to be interpreted inclusively rather than exclusively. Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, some exemplified preferred methods and materials are described herein.

The following Examples as well as the accompanying Figures are intended to provide illustrative embodiments of the present invention described and claimed herein. These Examples and Figures are not intended to provide any limitation on the scope of the invented subject-matter.

### BRIEF DESCRIPTION OF THE FIGURES

FIG. 1 shows a schematic overview of the formation of Vim3/Atg7/p53 complexes in the cell nucleus. In contrast to healthy, non-tumorous cells, in various tumor cells, in particular carcinoma cells, the endothelin B receptor (ETBR) is absent or downregulated. Therefore, endothelin-1 (ET-1) may trigger a strong signal via the endothelin A receptor (ETAR). This may lead to the formation of Vim3/Atg7/p53 complexes. These may then migrate to the cell nucleus and trigger certain transcription factors such as, e.g., focal adhesion kinase (FAK). This may have an influence on cellular signal transduction and tumor progression.

FIG. 2 shows serum levels of Vim3 polypeptide in blood serum obtained from individuals having different types of malignant carcinoma (Ca) determined by ELISA. It is visible that all types of tumors bear increased levels of Vim3 polypeptide over the control samples) ***p<0.001).

FIG. 3 shows serum levels of Vim3 polypeptide in blood serum obtained from individuals having prostate carcinoma (PCa) determined by ELISA. It is visible that the propensity to metastasize (PCa+Metastasis) and to form recidivism increases (PCa Rezidiv) the Vim3 serum levels, whereas a non-metastasizing prostate malignant tumors (PCa) merely shows a somewhat increased Vim3 polypeptide serum level. (n=30)

FIG. 4 shows fluorescence staining of prostate carcinoma tissue with a labelled Vim3-specific antibody and DAPI overlay demonstrating that there are aggregates (pits) containing Vim3 in the cell nuclei (indicated by the white arrows).

FIG. 5 shows urothelial carcinoma tissue (left: HE staining, right: Vim3 staining). The localization of Vim3 is mainly nuclear.

FIG. 6 shows small-cell carcinoma tissue (lung)(left: HE staining, right: Vim3 staining). The localization of Vim3 is mainly nuclear.

FIG. 7 shows mammacarcinoma tissue (upper row: parental tumor of ductal carcinoma); lower row: liver metastasis thereof) (left column: HE staining, right column: Vim3 staining). The patient has been treated with tamoxifen. The localization of Vim3 is mainly nuclear.

FIG. 8 shows mucoepidermiod carcinoma tissue. The localization of Vim3 is mainly nuclear.

FIG. 9 shows a myoepithelial adenoma (Vim3 staining). The staining in the nuclei is less prominent compared to the carcinoma.

FIG. 10 shows a pleomorphic adenoma (Vim3 staining). The staining in the nuclei is less prominent compared to the carcinoma.

FIG. 11 shows an oncocytoma (Vim3 staining) for comparative purposes. The staining of full-length vimentin (FL) as well as Vim3 is mainly cytosolic. The cell nuclei are visible as darker areas within the cells.

FIG. 12 shows an immunofluorescence staining of paraffin-embedded prostate cancer tissue samples. The upper row shows the staining of p53, the middle row the sating of Atg7 and the lower row the staining of Vim3. Depicted in the DAPI counterstain of the nuclei (left column), the specific protein staining (pg3, Atg7, Vim3) (middle column), and the overlay of both (right column).

FIG. 13 shows a wounding assay using prostate cell line DU145, demonstrating that Endothelin-1 (ET-1) stimulation with simultaneous endothelin B receptor (ETBR) blockade (BQ788) leads to rapid tumor proliferation closing the scraped area (via the endothelin A receptor (ETAR)) (cf., BQ788+ET-1).

FIG. 14 shows serum levels of endothelin (ET-1) expression in prostate carcinoma patients (Prostate Ca) versus healthy individuals (control).

FIG. 15 shows an immunohistochemical staining of full-length vimentin (VimFL) versus Vim3 in combination with an HE stain (HE) in healthy prostate tissue (upper row) versus prostate carcinoma (lower row).

FIG. 16 shows the mRNA Vim3 expression after tamoxifen treatment. In particular, ET-1 expression is triggered by tamoxifen treatment.

FIG. 17 shows results from a Western Blot analysis of nuclear extracts depicting the ER-alpha expression (left column), the ER-beta expression (middle column) and the ET-1 expression (right column). In particular, ET-1 expression is triggered by tamoxifen treatment.

FIG. 18 shows the immunoprecipitation with either ERα (A) or ET-1 antibody (B).

FIG. 19 shows the immunofluorescence ER-beta and ET-1.

FIG. 20 shows a Proximity Ligation Assay (PLA) assay of tamoxifen-treated (lower row) versus untreated cells (upper row).

FIG. 21 shows Vim3 levels in sera of individuals who are of different risks of having prostate cancer in comparison to control individuals. In the groups of higher risk, metastasis and recurrence, the level is elevated.

FIG. 22 shows Vim3 expression in serum of prostate cancer patients. In the groups of higher risk, metastasis and recurrence, the level is elevated. The horizontal line indicates an example of a threshold level of approximately 350 ng/ml between higher and lower risk.

FIG. 23 shows Vim3 levels in sera of individuals who are suffering from urothel carcinoma in comparison to control individuals. The presence of an urothel carcinoma increases Vim3 levels.

FIG. 24 shows Vim3 expression in serum of individuals who are suffering from urothel carcinoma in comparison to control individuals. The presence of an urothel carcinoma increases Vim3 expression. The horizontal line indicates an example of threshold level between 250 and 300 ng/ml between higher and lower risk.

FIG. 25 shows Vim3 expression analysis of treated MX-1 (a), (b), and MCF-7 (CC) cells by qRT-PCR analysis. In MX-1 cells, (a) shows the complete stimulation set-up where the paired t-tests have a significant difference when comparing stimulations. (b) shows the same without the T+ET-1 values to get details on the other stimulation settings which all represent significant different means after paired t-test. In both diagrams the increase in Vim3 expression up to ca 700 RFU upon a combinational stimulation with Tam is decisive. Data shown is derived from quadruplicates from an independent experiment. *P<0.05, **P<0.01 and ***P<0.001. Results from MCF-7 cells for Vim3 in (c) showing a clear increase in mRNA Vim3 expression after combined pretreatment with tamoxifen and 11-estradiol/ET-1; **p<0.01.

FIG. 26 shows the comparison of Vim3 expression after ET-1 vs. combined ET-1 plus Tamoxifen pretreatment. WB with 20 pg non-precipitated total cell extract of stimulated MCF-7. The corresponding densitometric analysis of the present bands of a protein analysis on the WB membrane where the intense band of Vim3 (47 kDa) is striking as compared to the very faint band of GAPDH in the same sample is shown with ImageJ, where the background and the control had been subtracted.

### EXAMPLES

### Example 1—Vim3 ELISA

Methods

ELISA plates were washed 2× in PBS, incubated with 100 μl of the patients serum for 2 hours at room temperature (RT), washed 2× with PBS, incubated with Vimentin 3B4 antibody for 1 h at RT, washed 2× with PBS, incubated again for 1 h at RT with Vim3 antibody, washed 2× with PBS and finally incubated with the secondary, peroxidase-labeled antibody for 1 h at RT. For the development of the ELISA 3,3′,5,5′-Tetramethylbenzidin (TMB solution) was used and reaction was stopped with sulfuric acid stop solution. The determination was done in an ELISA reader by a wavelength of 495 nm. For the quantification and control, high and low control was loaded on the same plate.

### Results

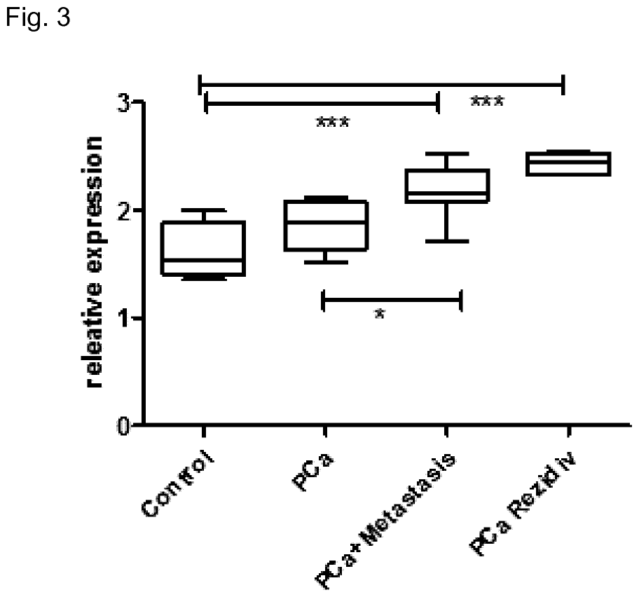

The results are depicted in FIGS. 2 and 3. It was found that all types of malignant tumors bear increased levels of Vim3 polypeptide over a control sample reflecting a healthy individual of the same species (cf., FIG. 2). In individuals in whom a prostate carcinoma (PC) has been found, it was surprisingly be found that the propensity to metastasize and to form recidivism increases the Vim3 serum levels, whereas a non-metastasizing prostate malignant tumors show less increase of Vim3 polypeptide serum level. These results indicate that the methods of the present invention are particularly useful for identifying malignant tumors, in particular malignant carcinoma in an individual.

### Example 2—Immune Histology and Immunofluorescence of Paraffin Embedded Tissues

### Methods

### Immune Histology.

Paraffin embedded tissue sections (3 μm thick) were deparaffinized by incubation for 2-5 min in xylene, followed by 2-3 minutes in 100% ethanol and 1 minute in 90% ethanol, and 1 min 70% ethanol, 1 min 50% ethanol and then rinsed with distilled water. The slides were incubated with a specific serum blocker (anti-rabbit) for 30 minutes in order to avoid nonspecific binding. After that incubation period, the slides were re-incubated for 1 hour at room temperature with specific primary antibodies (Vim3, EZBiolab, Inc. Carmel, USA, Vimentin V9, Santa Cruz, Heidelberg, Germany, AMACR and CD117, Dako, Hamburg, Germany). Following washes with PBS-Tween 20, the sections were incubated with a secondary, anti-rabbit antibody (Santa Cruz, Heidelberg, Germany). After rinsing with PBS-Tween 20, the slides were re-incubated for 2 min in 95% ethanol, followed by 2-3 minutes in 100% methanol, counterstained with an HE stain, and cover slipped.

### Immunofluorescence.

3 μm thick paraffin embedded tissue sections were deparaffinized by incubation for 1×5 min in xylene, followed by 1×5 min 100% ethanol and 1 min 70% ethanol, 1 min 50% ethanol and then rinsed with distilled water. After an incubation period in 5% PBS milk for 30 min, the slides were re-incubated for 1 hour at room temperature with specific primary antibodies (Vim3) in 3% PBS milk. Following washes with PBS, the sections were incubated with a secondary FITC-anti-rabbit antibody (Santa Cruz). Subsequent to rinsing with PBS, the slides were then counterstained with DAPI mounting medium (nuclear staining) and cover slipped.

### Results

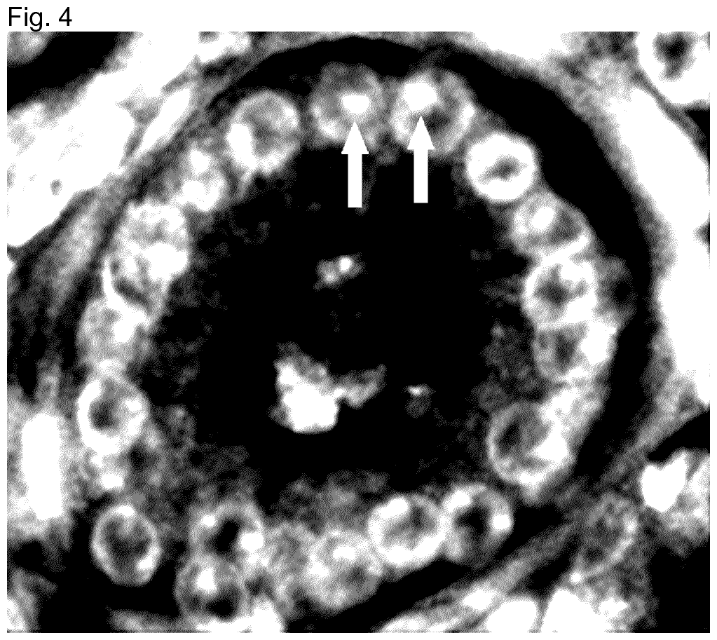

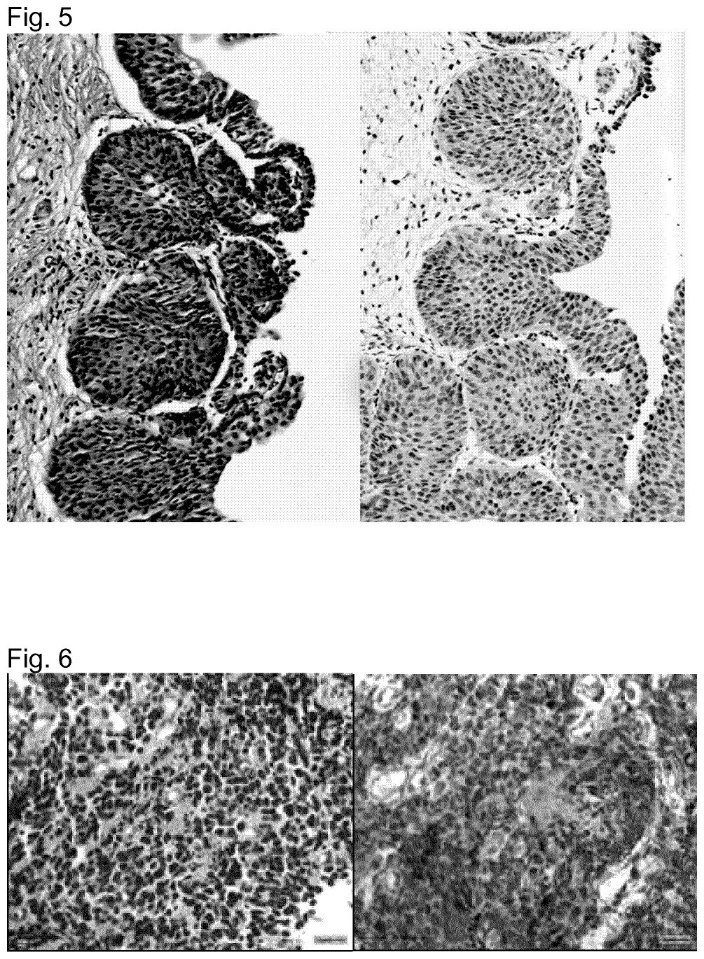

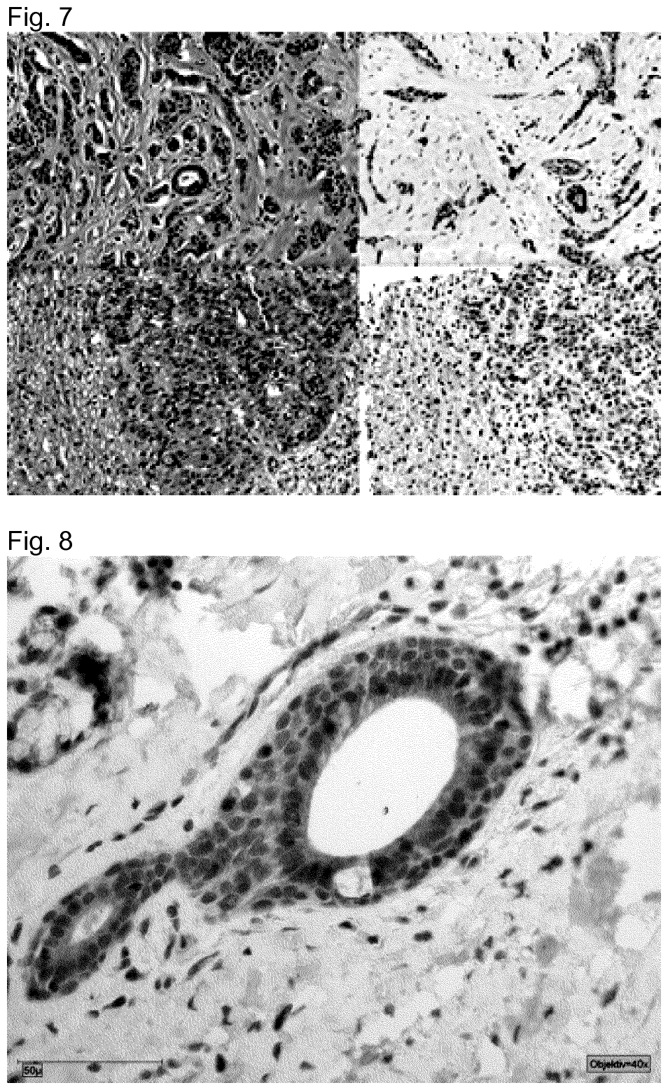

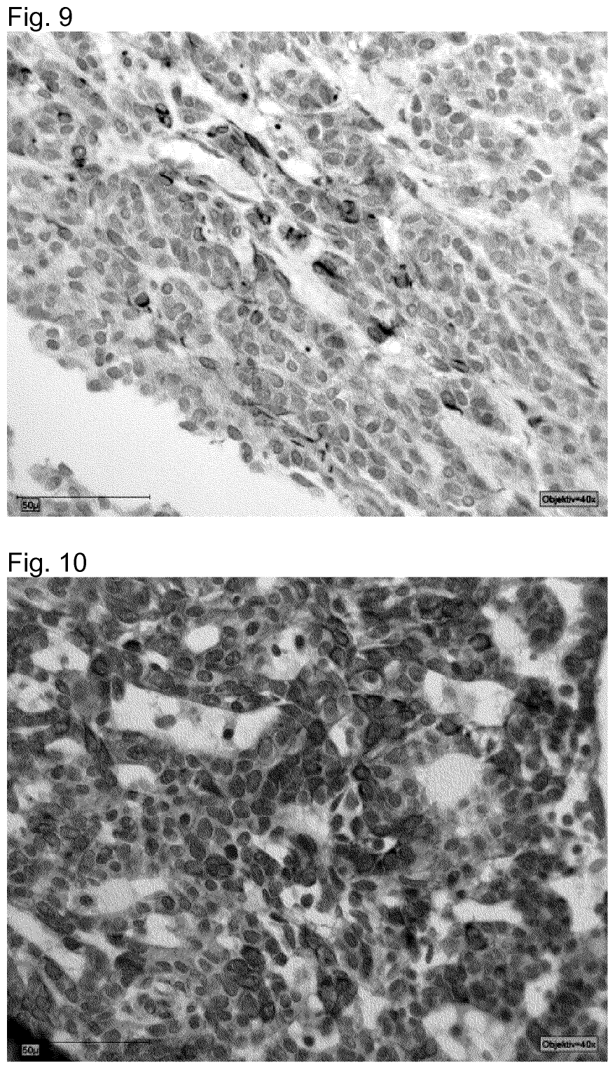

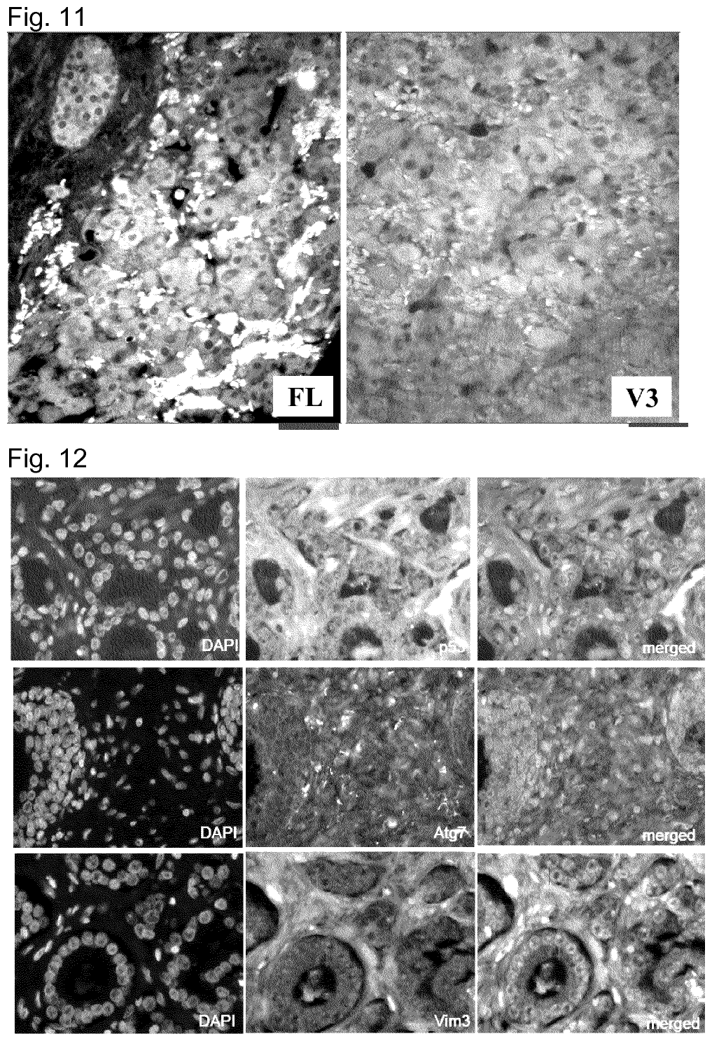

The results are depicted in FIGS. 4 to 11. It was found that in prostate carcinoma tissue aggregates (pits) containing Vim3 were located in the cell nuclei (cf., FIG. 4). It was further demonstrated that also in other malignant cancerous tissues, in particular carcinoma tissues, such as urothelial carcinoma tissue, in small-cell carcinoma tissue (lung), in mammacarcinoma tissue including metastases thereof, and in mucoepidermiod carcinoma tissue, the localization of Vim3 is mainly nuclear (cf., FIGS. 5 to 8). In contrast, in more benign tissues such as in neoplasia myoepithelial adenoma tissues and in pleomorphic adenoma tissues, the staining in the nuclei was found to be less prominent compared to the aforementioned carcinoma (cf., FIGS. 9 and 10). In benign oncocytoma, Vim3 is, in contrast to malignant tissues, mainly located cytosolic (cf., FIG. 11). These results indicate that the malignant tumors may also be identified by determining the intracellular localization of Vim3. Nuclear localization is a further indication of malignancy of the tissue.

### Example 3—ET-1 Expression in Serum Samples from Prostate Carcinoma Patients

Elevated ET-1 levels in prostate cancer patients have surprisingly been found to be a sign for higher metastatic potential of the tumors. It was found that in normal prostate cells a balance between vimentin, p53 and Atg7 exists. The involvement of ET-1 in the occurrence of metastases was analyzed via the activation of the vimentin, p53 and Atg7 pathway.

### Methods

In the collective of serum samples from patients with prostate cancer, significant upregulated ET-1 levels were found via ET-1 specific ELISA. Furthermore, cell culture experiments were conducted after ET-1 treatment over different time points and analyzed via Western blot the expression of the different proteins mentioned above. For the ET-1 ELISA and Vim3 ELISA 100 μl of serum was used. DU145 cells were cultured under normal conditions and treated with the substances mentioned. Cells were scratched before treatment. The ET-receptors were blocked 1 h before ET-1 stimulation. Immunofluorescence was performed on paraffin-embedded prostate cancer samples. This immunofluorescence has been carried out as described in Example 1 above. In addition to Vim3-specific antibodies, also Atg7- and pg3-specific primary antibodies have been used.

### Results

It could be shown in cell culture experiments that after ET-1 treatment the cells produce more Vimentin 3, a splicing product of full length Vimentin. Since the interaction between Vimentin, p53 and Atg7 does no longer exist and Vim3, p53 and Atg7 all translocate into the cell nucleus (cf., FIG. 12). Both factors, Athg7 and p53, are considered to build a complex with Vim3. Such Vim3/Atg7/p53 complex may indicate a tumor with higher degree of malignancy. These cells typically need a flexible cytoskeleton, so that they could easily change the shape and leave the cell layer. In prostate cancer, the ET-1 B receptor (ETBR) is frequently downregulated. It was found that in serum from prostate cancer patients the ET-1 level is increased. ET-1 was found to regulate Vim3 overexpression. Vim3 ELISAs of serums from patients with either prostate cancer, prostate cancer with metastasis, recurrent prostate cancer, and compared them with a control group, showed a significant increase of Vim3 expression in serum, especially in prostate cancer with metastasis and recurrence (cf., FIGS. 2 and 3).

### Example 4—Scratch-Assay

### Methods

The high metastasizing prostate cancer cell line DU145 was cultured under standard conditions (37° C. with 5% CO₂ and a humidity of 95%). For the performance of the experiments cells were grown on a 12-well plate until they reached a confluency of 80%-90%. Cells were starved in fetal calf serum- (FCS)-free medium for 24 hours. After starvation, the scratch was performed with a small clear pipette tip, area was marked. Cells were than treated with either 100 nM BQ123, an ET-1 A receptor (ETAR) antagonist or 100 nM of BQ788, an ET-1 B receptor (ETBR) antagonist, Bosentan (10⁴ M), Withaferin A (1 μM) 1 hour before ET-1 treatment or in case of withaferin and ETBR block, cells were incubated for 1 hour with both substances before ET-1 treatment for 6 hours or 24 hours. Scratch-Assay was analyzed after 3 hours, 6 hours, 12 hours, and 24 hours.

### Results

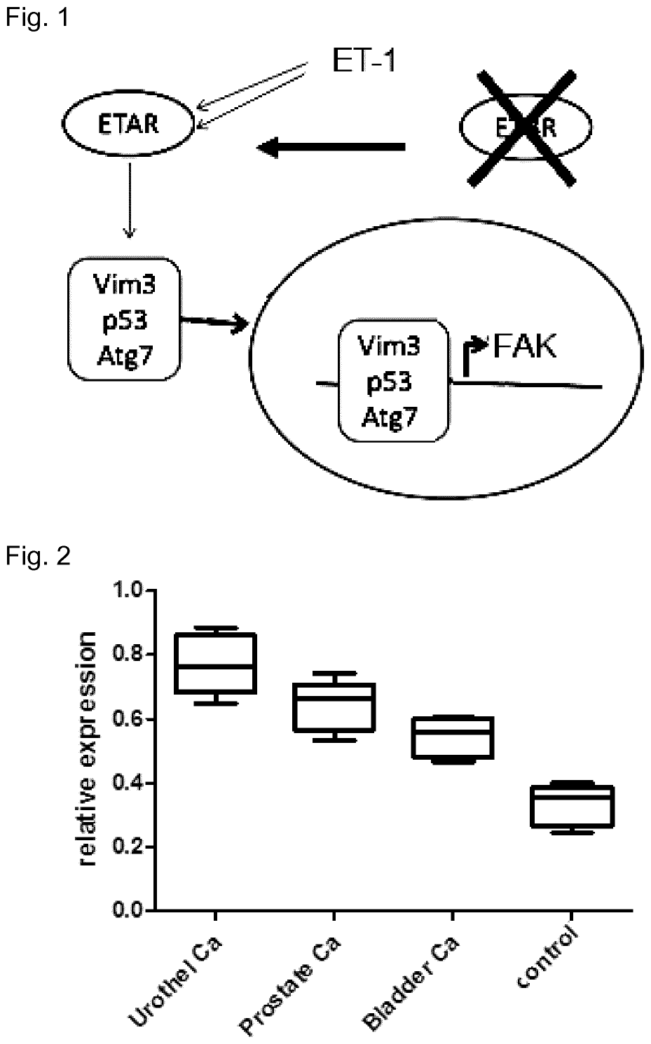

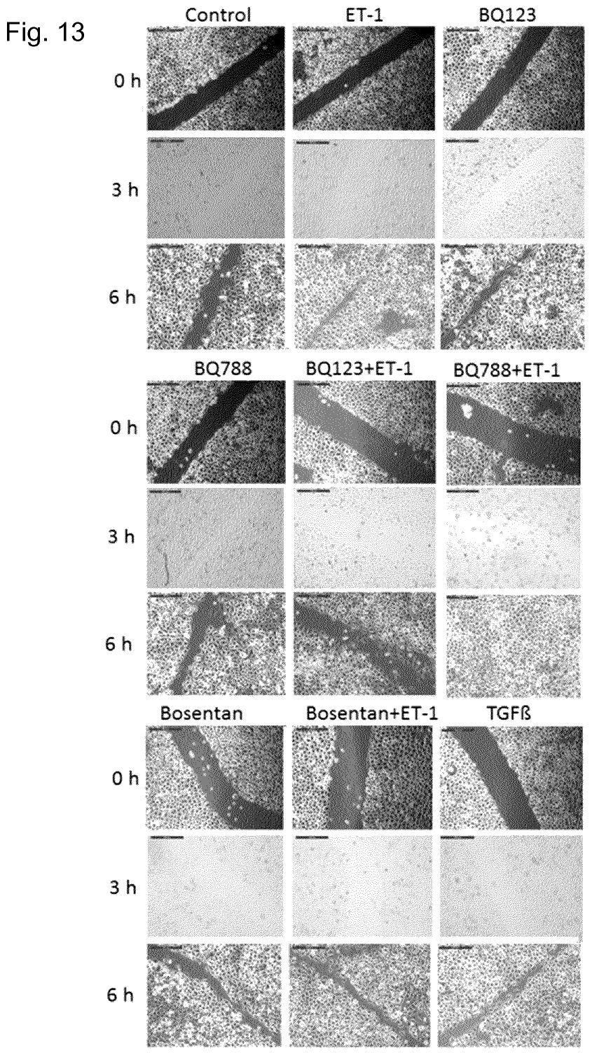

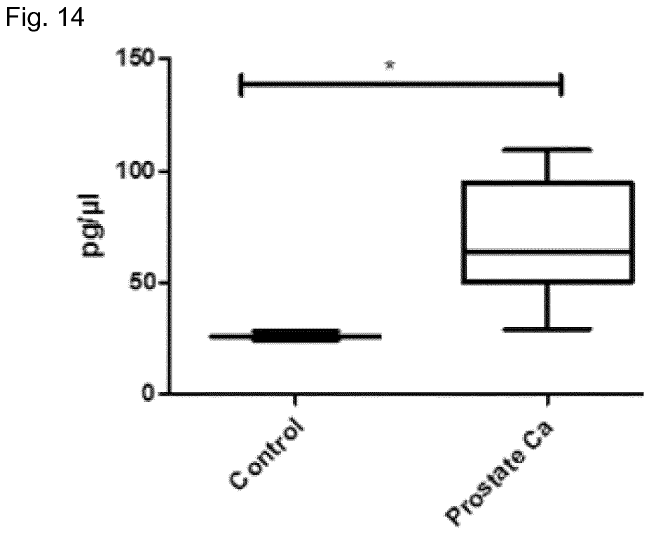

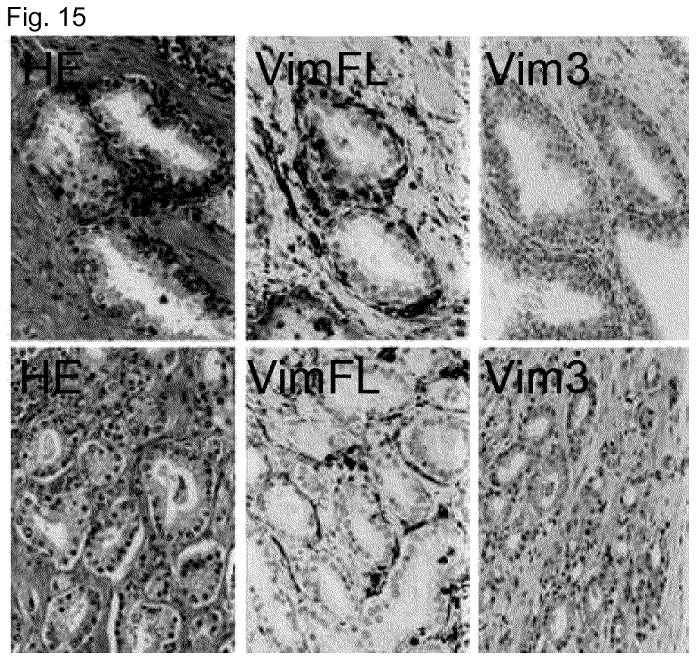

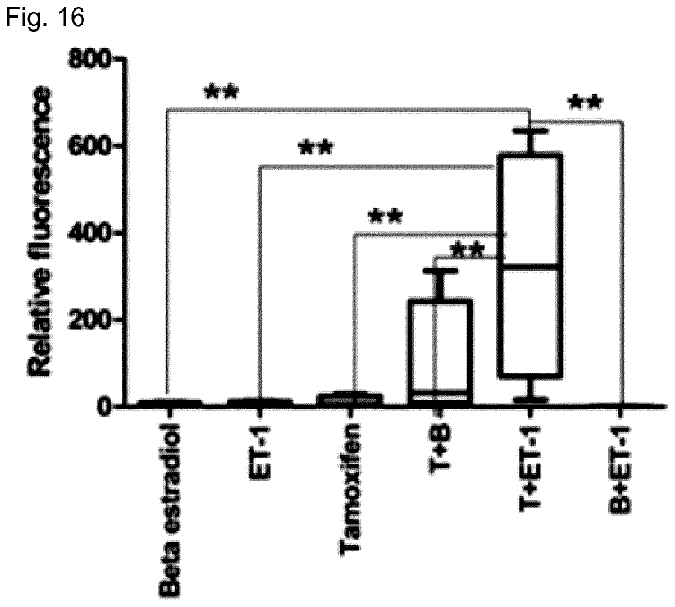

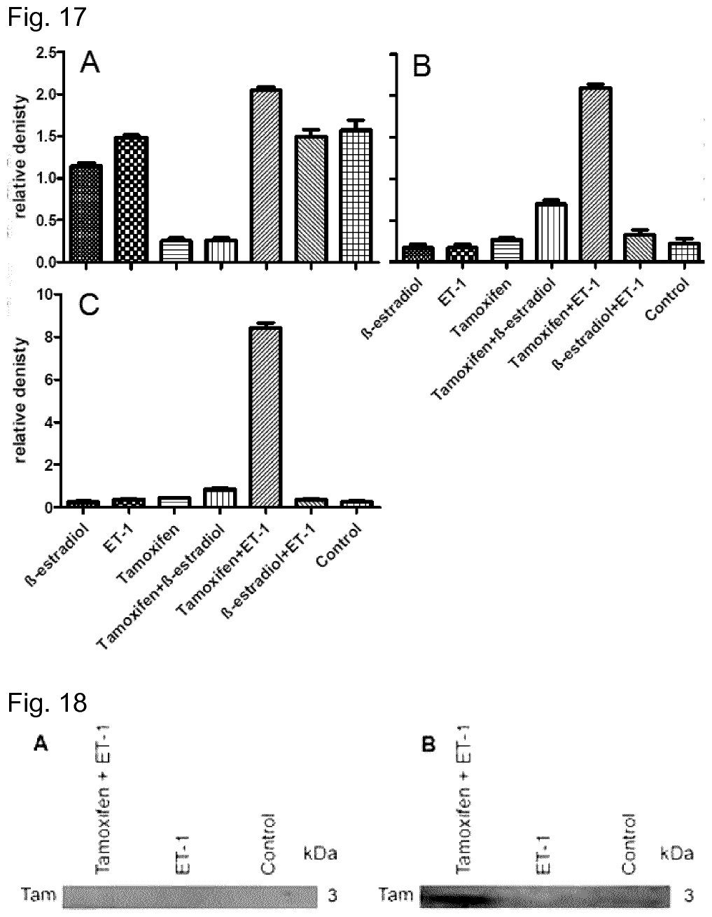

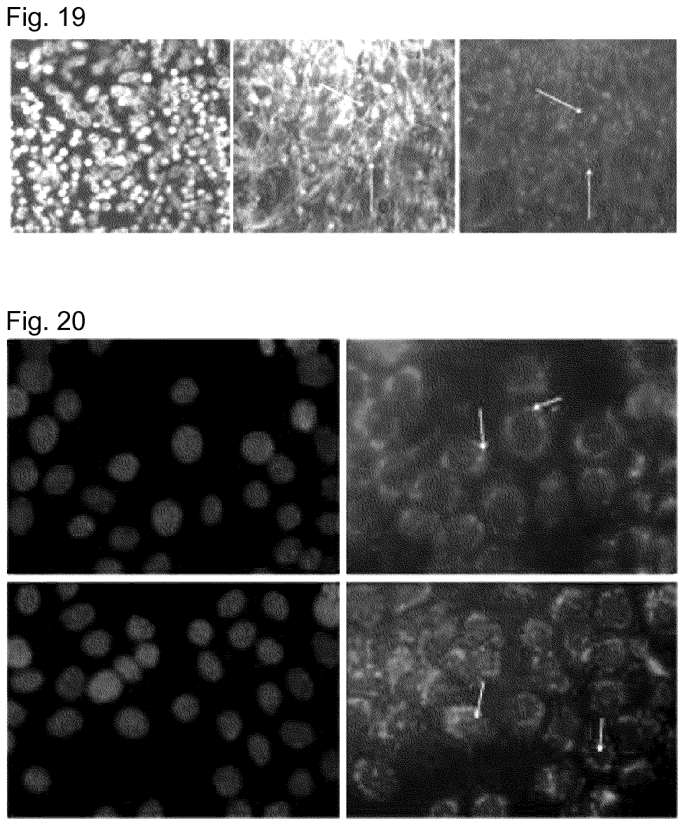

The results are also depicted in FIG. 13. It was found that the cellular signaling of the activation of cells (in particular their migration potential) triggered by endothelin-1 (ET-1) leads to particularly high growth activation of the cells when an ET-1 B receptor (ETBR) is blocked. Therefore, the route via activation of the ET-1 A receptor (ETAR) was found to lead to specifically high cellular growth induction. The respective molecular scheme is depicted in FIG. 1. Accordingly, blocking the ETBR in combination with Addition to ET-1 (cf., sample BQ788+ET-1) led to particularly high growth into the scratch. In this sample, after 6 hours the whole scratch was overgrowth. This finding confirms the molecular scheme as displayed in FIG. 1.

### Example 5—Anticancer Treatment in Correlation with Increased ET-1 Levels and Development of Metastases

Endothelin-1 was found to be responsible for the overproduction of Vim3. Vimentin in the full length version plays a role for the maintaining the cell structure and the anchorage of cells. It was of further interest to study the involvement of ET-1 in the development of metastatic breast cancers after chemotherapy.

### Methods

The dependency of the overexpression of Vim3 due to ET-1 presence and after tamoxifen treatment was shown in cell culture experiments and analyzed via Western blot. The expression of Vim3 and ET-1 was analyzed in paraffin-embedded tissue samples of patients with metastatic breast cancers after tamoxifen treatment. MCF-7 cells were cultured under normal conditions and treated with the mentioned substances. Tamoxifen treatment was performed 1 hour prior ET-1 or ß-estradiol treatment. Nuclear extracts were isolated and analyzed via Western blot under standard conditions. A Proximity Ligation Assay (PLA) assay was performed according to the manufacturer's protocol. From cells after treatment, the mRNA was isolated and qRT-PCR was performed. Immunohistochemistry was performed according to standardized conditions.

### Results

These experiments indicate that an anticancer agent like tamoxifen and ET-1 in combination is responsible for the overexpression of Vim3. The increased expression of ET-1 in combination with tamoxifen treatment of patients with cancer appears responsible for the development of metastases. In nuclear extracts from breast cancer cells, after treatment with either ET-1, beta-estradiol, Tamoxifen or a combination of them, significant upregulation of the ET-1 signal in the nuclear fraction after Tamoxifen treatment was detectable. The translocation of ET-1 into the nucleus is receptor. Immunoprecipitation showed that the translocation is not ER-alpha dependent. A Proximity Ligation Assay (PLA) assay showed that the ET-1 and the ER-beta build a complex. It was surpsisingly found that in case of tamoxifen-treated tumors, an increase of Vim3 was found in both, the tumor as well as the metastases, indicating that the translocation of ET-1 into the nucleus via ER-beta is responsible for the overproduction of Vim3, which may ease the cells to leave the cell layer. These findings further supports the finding that, first, the molecular mechanism laid out in FIG. 1 is correct and, second, that Vim3 levels are a well-usable indicator of malignant tumors and their degree of malignancy.

### Example 6—Vim3 Levels in Blood Sera of Patients Suffering from Various Cancers

ELISA plates were coated with solutions of 1:1000 3B4 antibody (directed against full-length Vimentin) for 1 h at RT, washed 2× with PBS, incubated 50 μl of patient's serum for 1 h at RT, washed 3× with PBS, incubated with Vim3 antibody for 1 h at RT, and subsequently incubated with TMB for 10 min at RT and treated with stop solution. Then, the samples were analyzed.

In these experiments, control individuals are such suffering from nephritis, bladder infection etc. Individuals of low risk are those with Gleason Scale (Gleason) 6. Individuals of intermediate risk are those with Gleason 7a and 7b. Individuals of high risk are those with Gleason 8, 9 or 10. Metastasizing prostate cancer is often under therapeutic treatment.

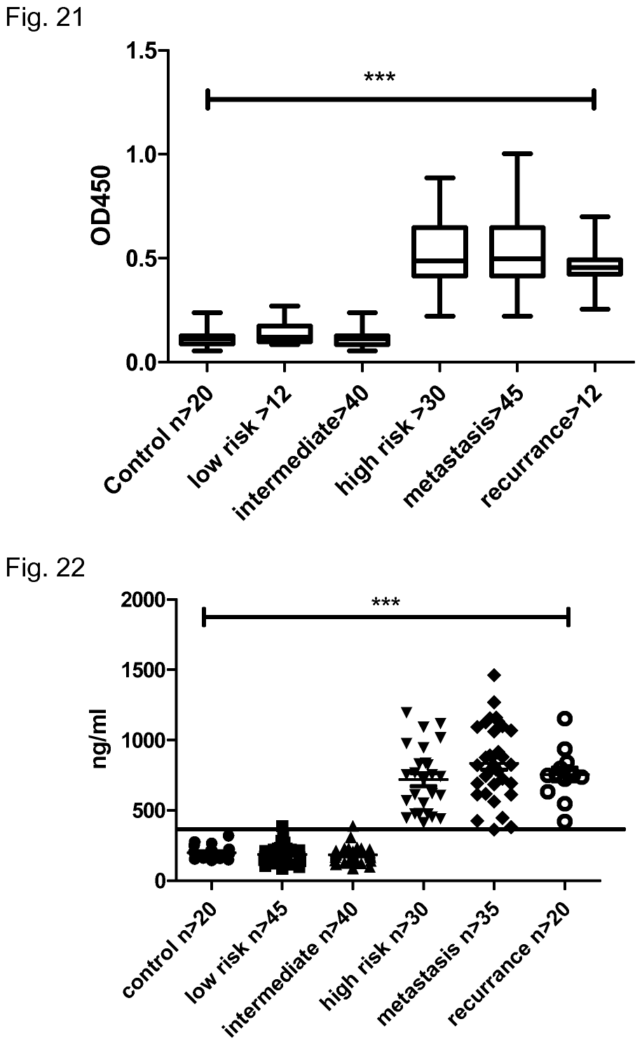

It was found that patients who are at higher risk of prostate cancer (prostate carcinoma), having metastases and/or recurrence show a significantly elevated Vim3 level (cf. FIGS. 21 and 22). It was found that, typically, a Vim3 level above 350 ng/ml is indicative for a rather high risk. A Vim3 level of 320 to 350 ng/ml is indicative for a still notable risk and a Vim3 level below 320 nm indicates a rather low risk.

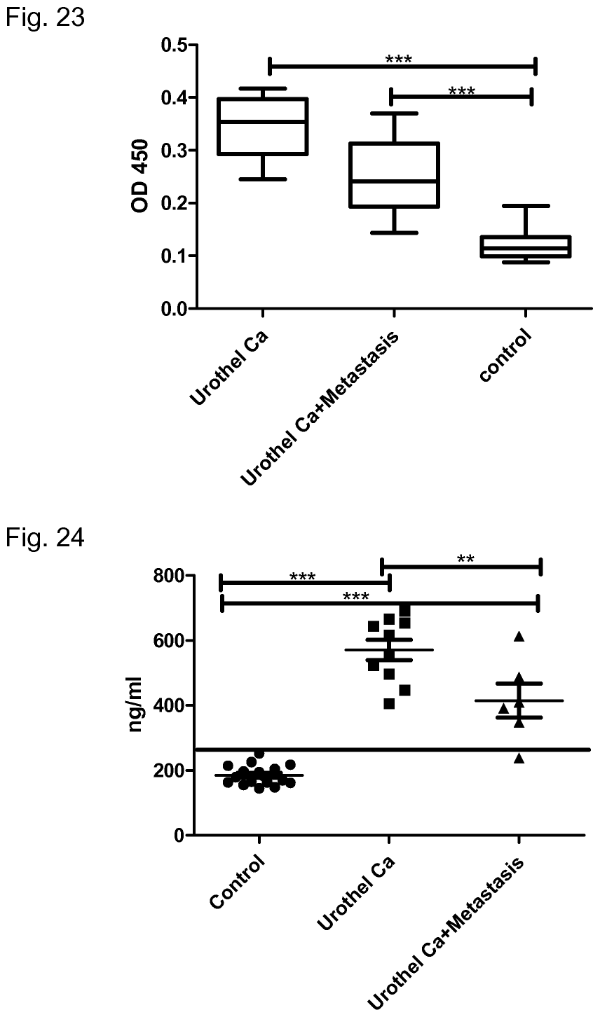

It was found that patients who are at higher risk of urothel carcinoma and optionally having metastases show a significantly elevated Vim3 level (cf. FIGS. 23 and 24). Interestingly, the presence of metastases decreased the Vim3 level, although it still remains clearly above the control level. It was found that, typically, a Vim3 level above 300 ng/ml is indicative for a rather high risk. A Vim3 level of 250 to 300 ng/ml is indicative for a still notable risk and a Vim3 level below 300 nm indicates a rather low risk. Also in these experiments, control individuals are such suffering from nephritis, bladder infection etc.

It was found that patients who are at higher risk of pancreas carcinoma and optionally having metastases show a significantly elevated Vim3 level (data not shown). The threshold levels are in a comparable range as shown above for prostate cancer (prostate carcinoma) and urothel carcinoma.

### Example 7—Vim 3 in Breast Cancer

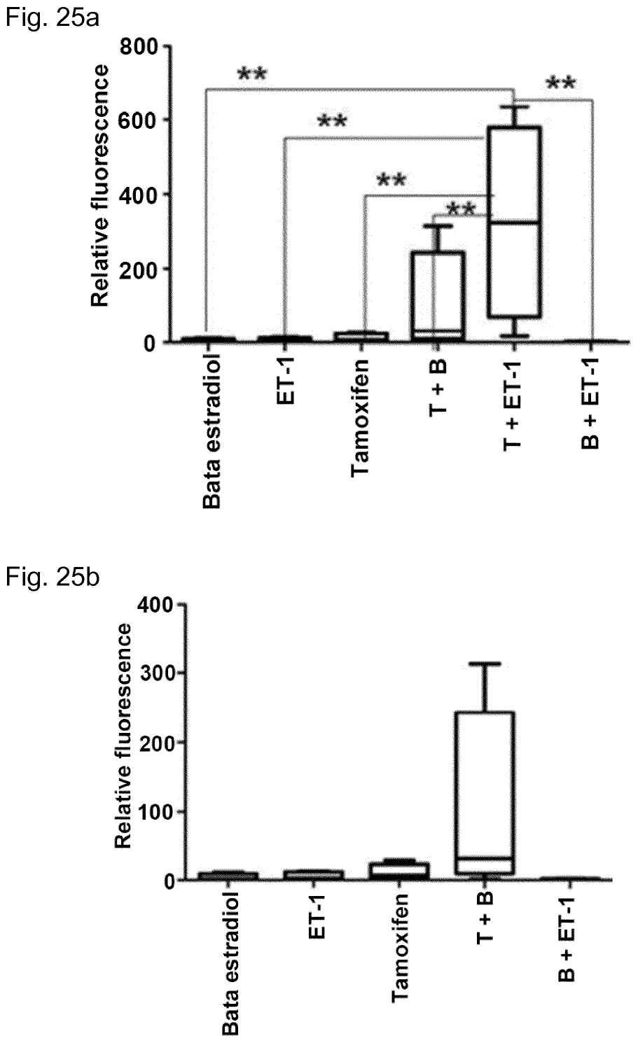

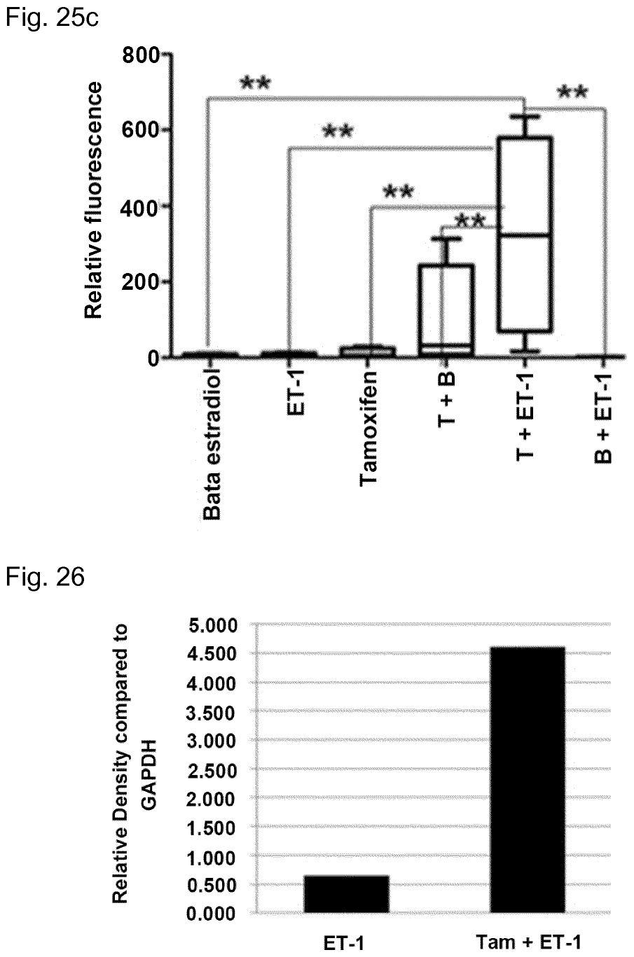

As the PCR showed unexpected results of ERß transcripts in MX-1, protein levels were investigated by Western blot. FIG. 25(a) shows the results of 20 pg nuclear extract of stimulated MX-1 probed with ERβ, ET-1 and Lamin B antibody (as loading control). ERβ was found after all different stimulations, with slightly varying intensity.

Furthermore, the effect of Tam was studied with regard to its translational capacity for Vim3 after a combined Tam plus ET-1 pretreatment versus ET-1 alone. As the analysis of the Western blot depicts by densitometry (FIG. 26), adding Tam as additional mediator to ET-1 results in highly statistically significant induced formation of Vim3 (***p<0.001). Tam upregulates Vim3.

Vim3 Expression in Invasive Ductal Breast Carcinoma, and Its Metastases From the pre-evaluation of breast cancer specimen via tissue-microarray it was known that ET-1 and Vim3 staining could be detected in tumor cells, while vimentin full length was only positive in the interstitial tissue. To analyze the expression of the newly described target gene Vim3 in more detail, its expression was investigated in 28 biopsies and their respective resection specimens of breast cancer cases. All biopsies showed grade 2 invasive carcinoma positive for ERα expression. In each biopsy, Vim3 was detected, either in nuclear (20/25; strong expression: 3+) and/or diffuse cytoplasmic (25/25) localization. After Tam treatment, seventeen respective resection specimens were still diffusely positive for Vim3, now mainly in a cytoplasmic localization (12/17), with nuclear localization additionally found in 5 specimens (5/17). In 4 cases, lymph node but no other metastases were reported (4/28). In contrast, 7 cases developed organ metastases (7/28), of which 3 had additional lymph node metastases.

The expression of Vim3 and ERß was investigated in resection specimens and their metastases. In a set of 8 cases positive for ERα after tam pretreatment, it was found again a diffuse cytoplasmic expression of Vim3. In addition, in 10/12 metastases (pleura 2×, skin 2×, bone 3×, uterus 1×, liver 1×, soft tissue 1×) Vim3 was also expressed (cytoplasm: 12/12; additional nuclear expression 5/12). In 7 of the 12 metastases, a 3+ nuclear expression for ERR was found, which was in all investigated cases accompanied by Vim3 expression. It was found that almost all biopsies show a nuclear positivity for Vim3 (19/21), which is changed to a cytoplasmic staining in mastectomies after Tam (13/17). These data regarding Vim3 in breast cancer are described more in detail in von Brandenstein et al., Journal of Cancer Therapy, 2018, 9, 438-463.

1. A method for detecting a malignant tumor in an individual, said method conducted in vitro comprising the following steps:
(i) providing blood serum S obtained from the individual; and
(ii) determining the Vimentin variant 3 (Vim3) level in the blood serum S,
wherein an increased Vim3 level in the blood serum S indicates the presence of a malignant tumor in the individual.

2. The method of claim 1, wherein the malignant tumor is malignant carcinoma.

3. The method of claim 1, wherein the malignant tumor is a malignant carcinoma selected from the group consisting of prostate carcinoma, urothelial carcinoma, bladder carcinoma, transitional cell carcinoma, mucoepidermiod carcinoma, mammacarcinoma, small-cell carcinoma, myoepithelial carcinoma, adenocarcinoma, gastric signet ring cell carcinoma, and esophageal carcinoma.

4. The method of claim 1, wherein an increased Vim3 level in the blood serum S indicates the presence of one or more metastases in the individual, the propensity of the tumor to metastasize, or a combination of both.

5. The method of claim 1, wherein the method further comprises step (iii) of comparing the Vim3 level determined in step (ii) with a predetermined reference value R1 indicating the borderline between blood serum indicating the presence of a malignant tumor and a blood serum of the same species indicating the absence of a malignant tumor,
wherein an Vim3 level determined in the blood serum S that is higher than R1 indicates the presence of a malignant tumor in the individual, and
wherein the Vim3 level is related to the total polypeptide content comprised in the respective sample.

6. The method of claim 1, wherein the method further comprises step (iii) of comparing the Vim3 level determined in step (ii) with a Vim3 level determined in a control sample C obtained from one or more control individuals of the same species free of a malignant tumor,
wherein an Vim3 level determined in the blood serum S that is at least 10% higher than the Vim3 level of C indicates the presence of a malignant tumor in the individual, and
wherein the Vim3 level is related to the total polypeptide content comprised in the respective sample.

7. The method of claim 1, wherein the step (ii) of determining the Vim3 level is determining the level of Vim3 polypeptide.

8. The method of claim 1, wherein the step (ii) of determining the Vim3 level is determining the level of Vim3 polypeptide by means of conducting at least one step selected from the group consisting of enzyme-linked immunosorbent assay (ELISA), immuno-electrophoresis, immuno-blotting, Western blot, SDS-PAGE, capillary electrophoresis (CE), spectrophotometry or enzyme assay for example, dipsticks (lateral flow), and combinations of two or more thereof.

9. The method of claim 1, wherein the step (ii) of determining the Vim3 level is determining the level of Vim3 polypeptide and wherein said step (ii) comprises staining of the Vim3 polypeptide.

10. The method of claim 1, wherein the step (ii) of determining the Vim3 level is determining the level of Vim3 messenger RNA.

11. The method of claim 1, wherein the step (ii) of determining the Vim3 level is determining the level of Vim3 messenger RNA by means of conducting at least one step selected from the group consisting of polymerase chain reaction (PCR), real time PCR (RT-PCR), by in situ hybridization, gel electrophoresis, Southern Blot, and combinations of two or more thereof.

12. A method for detecting a malignant tumor in an individual, said method conducted in vitro comprising the following steps:
(i) providing a solid tissue sample T obtained from the individual; and
(ii) determining the intracellular localization of Vim3 polypeptide in the solid tissue sample T,
wherein an accumulation of Vim3 polypeptide in the cell nuclei in the solid tissue sample T indicates the presence of a malignant tumor in the individual.

13. A method for treating an individual bearing a malignant tumor, wherein the presence of the malignant tumor in the individual has previously been detected in the individual by means of a method of claim 1, wherein the individual is administered with a sufficient amount of an antineoplastic agent.

14. A method for treating an individual bearing a malignant tumor, wherein said malignant tumor is endothelin B receptor negative, wherein the individual is administered with a sufficient amount of an antineoplastic agent that is or comprises a Vim3 inhibitor.

15. The method of claim 13, wherein said antineoplastic agent is selected from the group consisting of withaferin A and Vim3 siRNA.

16. The method of claim 14, wherein said antineoplastic agent is selected from the group consisting of withaferin A and Vim3 siRNA.

17. The method of claim 1, wherein the step (ii) of determining the Vim3 level is determining the level of Vim3 polypeptide and wherein said step (ii) comprises staining of the Vim3 polypeptide, by means of:
(i) direct immunodetection comprising providing at least one labeled antibody or antibody fragment AB1-L specific for the Vim3 polypeptide, and
enabling the binding of said AB1-L to the Vim3 polypeptide; or
(ii) indirect immunodetection comprising providing at least one unlabeled antibody or antibody fragment AB1-ul specific for the Vim3 polypeptide and at least one labeled antibody or antibody fragment AB2-L specifically binding to AB1-ul,
enabling the binding of AB1-ul to the Vim3 polypeptide, and
enabling the binding of AB2-L to AB1-ul.


SEQUENCE LISTING (5 sequences)

SEQ ID NO: 1 - protein, 431 aa, Homo sapiens
MSTRSVSSSSYRRMFGGPGTASRPSSSRSYVTTSTRTYSLGSALRPSTSRSLYASSPGGVYATRSSAVRLRSSVPGVRLLQDSVDFSLADAINTEFKNTRTNEKVELQELNDRFANYIDKVRFLEQQNKILLAELEQLKGQGKSRLGDLYEEEMRELRRQVDQLTNDKARVEVERDNLAEDIMRLREKLQEEMLQREEAENTLQSFRQDVDNASLARLDLERKVESLQEEIAFLKKLHEEEIQELQAQIQEQHVQIDVDVSKPDLTAALRDVRQQYESVAAKNLQEAEEWYKSKFADLSEAANRNNDALRQAKQESTEYRRQVQSLTCEVDALKGTNESLERQMREMEENFAVEAANYQDTIGRLQDEIQNMKEEMARHLREYQDLLNVKMALDIEIATYRKLLEGEESRISLPLPNFSSLNLRGKHFISL

SEQ ID NO: 2 - protein, 8 aa, Homo sapiens
RGKHFISL

SEQ ID NO: 3 - protein, 10 aa, Homo sapiens
NLRGKHFISL

SEQ ID NO: 4 - DNA, 20 bp, Artificial Sequence
  Vim3 primer forward
gagaactttgccgttgaagc

SEQ ID NO: 5 - DNA, 23 bp, Artificial Sequence
  Vim3 primer reverse
gaaataaaatgcttacccctcag